(19) United States
(12) Patent Application Publication
(10) Pub. No.: US 2023/0270677 A1
(43) Pub. Date: Aug. 31, 2023

(54) LONG-ACTING THERAPEUTIC AGENT COMBINATIONS AND METHODS THEREOF

(71) Applicant: University of Washington, Seattle, WA (US)

(72) Inventor: Rodney J.Y. Ho, Seattle, WA (US)

(73) Assignee: University of Washington, Seattle, WA (US)

(21) Appl. No.: 17/791,504

(22) PCT Filed: Jan. 7, 2021
(86) PCT No.: PCT/US2021/012538

§ 371 Date: Jul. 7, 2022

Related U.S. Application Data
(60) Provisional application No. 62/959,077, filed on Jan. 9, 2020.

(51) Int. Cl. A61K 9/16; A61K 31/4985; A61K 31/506; A61K 31/7076; A61K 31/536; A61K 31/505; A61K 31/426; A61K 31/52; A61K 9/00; A61P 31/18

(52) U.S. Cl. CPC A61K 9/1617; A61K 31/4985; A61K 31/506; A61K 31/7076; A61K 31/536; A61K 31/505; A61K 31/426; A61K 31/52; A61K 9/0019; A61P 31/18

(57) ABSTRACT

The present disclosure describes simple, stable, and scalable antiviral therapeutic agent compositions that transform short-acting antiviral (e.g., anti-HIV) therapeutic agents that would otherwise require daily short-acting oral administration into long-acting injectable forms that lasts for many weeks per administration. A mixture of water-soluble and water-insoluble antiviral therapeutic agents can be present in the long-acting and drug-combination composition.

### CROSS-REFERENCE(S) TO RELATED APPLICATION(S)

This application claims the benefit of U.S. Patent Application No. 62/959,077, filed Jan. 9, 2020, the disclosure of which is incorporated herein by reference in its entirety.

### STATEMENT OF GOVERNMENT LICENSE RIGHTS

This invention was made with government support under Grant No. AI120176, awarded by the National Institutes of Health. The government has certain rights in the invention.

### BACKGROUND

About 37 million people globally are living with human immunodeficiency virus or HIV (people living with HIV “PLWHIV”), of which about 75% know their HIV infection status and about 86% are HIV-virally suppressed. Even among those on antiretroviral therapy (ART), only about 86% experience sustained viral suppression; hence 14% remain unsuppressed. Regardless, a majority of PLWHIV (˜35.7 million) live in low to middle income countries (LMICs). Due in part to expanded access to effective combination anti-infective drug combination ART, global HIV incidence and mortality is declining But PLWHIV must still take an oral daily dose of combination drugs for the rest of their lives. In essence, combination ART or cART has allowed HIV-positive people to live longer, healthier lives, and can eliminate HIV transmission risk from those who adhere to daily oral treatment.

However, cART is effective only when adherence is high and, consequently, HIV viral load is low. Currently, even for those who have access to combination HIV therapies, 14-19% exhibit detectable virus levels while on oral daily treatment. Non-adherence leads to viral rebound, increased drug resistance, progression to advanced HIV disease, and death. In addition, detectable plasma HIV (due to lack of awareness of status, but also for 14% of patients under cART care) poses significant challenges for reducing HIV incidence. Many people on oral daily regimens (up to two thirds) stop treatment for periods of up to six months and become at risk for developing advanced HIV disease. Patient adherence and continued care in LMICs is poor. While the reasons for non-adherence are multi-faceted, they include pill fatigue, forgetting to take daily pills, and stigma associated with carrying prescription bottles in social settings. A long-acting (LA) and potentially more effective injectable could improve equity by increasing the number of people initiating treatment after diagnosis, by reducing stigma associated with treatment, and by improving adherence and retention in care. An LA dosage form will also help prevent drug resistant viruses from emerging.

The attempt to develop a long-acting regimen composed of combination HIV drugs (or a complete regimen in one dosage form) is challenging for a number of reasons. The disparate chemistries typically present within different active pharmaceutical ingredients (APIs) prevent multiple drug combination that include water-soluble and water-insoluble drugs from staying together in an injectable suspension or dosage form. Specifically, co-formulation of hydrophobic and hydrophilic drugs into one regimen that exhibits desirable pharmacokinetics (PK) for all APIs has been a significant hurdle in the drug-development process. Thus, the existing approach for confronting this problem is to either modify the parent drug chemistry to change water solubility or conjugating amino or ester hydrophobic moieties to bring multiple drugs in one aggregate to form drug-combination aggregates. However, these approaches can modify the parent drugs' pharmacological and toxicological potency and properties, requiring additional time and capital investments in the drug-development process. Indeed, making a water-insoluble derivative from a water-soluble drug that was previously shown to be active in treating patients often leads to reduced potency compared to the water-soluble drug. Furthermore, developing one such combination regimen (without modifying the parent drug's pharmaceutical profile) that also exhibits long-acting PKs and persistent API levels in anatomical sites of HIV persistence, while desirable, has been very difficult to achieve without chemical modification of one of more drugs in the drug combination which has been proven to be effective in clinical use. Another barrier to the development of LA cART therapies is a perceived lack of commercial opportunities due to limited market potential. For the reasons cited above, there is no currently approved all-in-one long-acting combination anti-infective therapy for PLWHIV. Instead, when LA drug combination therapy is needed, two different formulations are typically given in two different injections, which exhibit different and asynchronous pharmacokinetics and cell distribution properties.

The current preferred HIV regimen, as per WHO latest recommendations in July 2019, is the drug combination TLD (water-soluble hydrophilic TDF or prodrug of tenofovir (T); lamivudine (L) (also known as 3TC); and practically water-insoluble hydrophobic dolutegravir (D)). Tolerability and issues related to durable viral suppression can be addressed by the adoption of oral TLD, although issues related to the variability of dosing, incomplete adherence, and other uptake and retention issues observed with oral pills will remain barriers in global HIV treatment.

Existing formulation technologies based on polymeric and liposomal delivery systems are either not suitable for keeping hydrophobic and hydrophilic drugs together or exhibited poor stability and loading. Without wishing to be bound by theory, it is believed that while lipid excipients with seemingly similar structures and composition could be used to make liposome or lipid nanoparticles, the specific assembly process and conditions as well as the inclusion of the APIs can lead to unique physically-assembled products that are drastically different in structure and/or properties.

A composition and process that allow hydrophobic and hydrophilic drugs to assemble in small drug combination particles suitable for producing stable, all-in-one cART injectable dosages in suspension is needed. The present disclosure fulfils these needs and provides further advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features an injectable aqueous dispersion, including an aqueous solvent, and an antiviral therapeutic agent composition dispersed in the aqueous solvent to provide the injectable aqueous dispersion. The antiviral therapeutic agent composition includes a combination of antiviral therapeutic agents selected from: dolutegravir, lamuvidine, and tenofovir and prodrugs thereof; efavirenz, lopinavir, and tenofovir and prodrugs thereof; lopinavir, ritonavir, lamuvidine, tenofovir and prodrugs thereof; efavirenz, tenofovir disoproxil fumarate, and emtricitabine (FTC); dolutegravir, tenofovir disoproxil fumarate, and emtricitabine; dolutegravir, lamuvidine, and tenofovir disoproxil fumarate; dolutegravir, lamuvidine, and abacavir; dolutegravir, lamuvidine, tenofovir and prodrugs thereof, and rilpivirine. The antiviral therapeutic agent composition further comprising one or more compatibilizers comprising a lipid, a lipid conjugate, or a combination thereof. The injectable aqueous dispersion exhibits a therapeutically effective plasma concentration of the combination of antiviral therapeutic agents for 2 or more weeks.

In another aspect, the present disclosure features a method of treating diseases caused by retroviruses, including parenterally administering to a subject in need thereof, at a frequency of at most one dose every 2 weeks, an injectable aqueous dispersion of the present disclosure.

In yet another aspect, the present disclosure features a powder composition including a combination of antiviral therapeutic agents selected from: dolutegravir, lamuvidine, and tenofovir and prodrugs thereof; efavirenz, lopinavir, and tenofovir and prodrugs thereof; lopinavir, ritonavir, lamuvidine, tenofovir and prodrugs thereof; efavirenz, tenofovir disoproxil fumarate, and emtricitabine (FTC); dolutegravir, tenofovir disoproxil fumarate, and emtricitabine; dolutegravir, lamuvidine, and tenofovir disoproxil fumarate; dolutegravir, lamuvidine, and abacavir; dolutegravir, lamuvidine, tenofovir and prodrugs thereof, and rilpivirine. The powder composition further includes one or more compatibilizers including a lipid, a lipid conjugate, or a combination thereof. The powder composition exhibits a therapeutically effective plasma concentration of the combination of antiviral therapeutic agents for 2 or more weeks.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a series of graphs showing long-acting plasma and cell (PBMC or peripheral blood mononuclear cell or lymphocytes) time-course of an embodiment of a composition including a combination of antiviral therapeutic agents of the present disclosure (TLD: Tenofovir TFV, Lamivudine 3TC, and Dolutegravir DTG; formulated in a DcNP dosage form). After a single subcutaneous injection of TLD assembled in a drug combination nanoparticle or DcNP (10 mg/kg each) dosage, the plasma (dark circles) and PBMC (light circles) levels of DTG, TFV and 3TC were measured until 4 weeks (672 hr). Data expressed were mean±SD of n=4 NHPs. Higher PBMC (light circles) to plasma (dark circles) exposure is notable. The dark lines in the three panels indicate EC₅₀ estimate for HIV each drug in the TLD composition. EC₅₀ values are from Tivacay (DTG or D), Viread (TFV, tenofovir or T), and Epivir (3TC, lamivudine or L) product labels for HIV-1.

FIG. 2 is a graph showing plasma concentration-time profiles of lopinavir, ritonavir, and tenofovir in macaques following a single subcutaneous dose of the antiviral therapeutic agents in either the soluble (free, open circles and dotted lines) or in an injectable aqueous dispersion of the present disclosure DcNP dosage form (closed circles and solid line).

FIG. 3 is a series of graphs showing the effect of the varying composition of compatibilizers on the plasma drug concentration over time of one of the 3 therapeutic agents called tenofovir (TFV) when administered to macaques as an injectable aqueous dispersion of the present disclosure (as part of a combination of 3 therapeutic agents).

### DETAILED DESCRIPTION

The present disclosure describes simple, stable, and scalable antiviral therapeutic agent compositions, such as drug combination nanoparticles (DcNP), that transform antiviral (e.g., anti-HIV) therapeutic agents that would otherwise require daily short-acting oral administration into long-acting injectable forms that last for many weeks per administration. The long-acting nature of the combinations of antiviral therapeutic agents of the present disclosure can be seen by the long acting plasma and cell concentrations of each of the antiviral therapeutic agents in non-human primates. Long-acting compositions including combinations of 2 to 4 antiviral therapeutic agents per dosage form can be made. Importantly, a mixture of water-soluble and water-insoluble antiviral therapeutic agents, which are generally incompatible and cannot be formed into a single unified composition, can be formulated together to provide long-acting injectable dosage forms, which exhibit sustained plasma levels for all the antiviral therapeutic agents in the composition. The long-acting injectable dosage forms can be used to improve patient adherence because chronic daily dosing often leads to poor patient compliance from pill fatigue. In turn, adherence can provide sustained therapeutic effects, particularly to sustain HIV suppression to prevent patients from progressing into AIDS and death.

Without wishing to be bound by theory, it is believed that the stable assembly of otherwise incompatible water-soluble and water-insoluble antiviral therapeutic agents is facilitated by lipid excipients through a well-defined formulation process. This unique drug-combination platform technology, called a drug combination nanoparticle (DcNP), could stabilize water-insoluble and water-soluble antiviral (e.g., antiretroviral) drugs in an injectable long-acting suspension intended to replace daily oral cART, which could help greatly with patient adherence.

### Definitions

At various places in the present specification, groups or ranges are described. It is specifically intended that the disclosure include each and every individual sub-combination of the members of such groups and ranges.

The verb “comprise” and its conjugations, are used in the open and non-limiting sense to mean that items following the word are included, but items not specifically mentioned are not excluded.

“About” in reference to a numerical value refers to the range of values somewhat less or greater than the stated value, as understood by one of skill in the art. For example, the term “about” could mean a value ranging from plus or minus a percentage (e.g., ±1%, ±2%, or 5%) of the stated value. Furthermore, since all numbers, values, and expressions referring to quantities used herein are subject to the various uncertainties of measurement encountered in the art, unless otherwise indicated, all presented values may be understood as modified by the term “about.”

As used herein, the articles “a,” “an,” and “the” may include plural referents unless otherwise expressly limited to one-referent, or if it would be obvious to a skilled artisan from the context of the sentence that the article referred to a singular referent.

Where a numerical range is disclosed herein, such a range is continuous, inclusive of both the minimum and maximum values of the range, as well as every value between such minimum and maximum values. Still further, where a range refers to integers, every integer between the minimum and maximum values of such range is included. In addition, where multiple ranges are provided to describe a feature or characteristic, such ranges can be combined. That is to say that, unless otherwise indicated, all ranges disclosed herein are to be understood to encompass any and all subranges subsumed therein. For example, a stated range of from “1 to 10” should be considered to include 1 and 10, and any and all subranges between the minimum value of 1 and the maximum value of 10. Exemplary subranges of the range “1 to 10” include, but are not limited to, e.g., 1 to 6.1, 3.5 to 7.8, and 5.5 to 10.

As used herein, the term “matrix” denotes a solid mixture composed of a continuous phase, and one or more dispersed phase(s) (e.g., particles of the pharmaceutically active agent).

The terms “therapeutic agent”, “active agent”, “drug”, and “active pharmaceutical ingredient” are used interchangeably herein.

As used herein, “biocompatible” refers to a property of a molecule characterized by it, or its in vivo degradation products, being not, or at least minimally and/or reparably, injurious to living tissue; and/or not, or at least minimally and controllably, causing an immunological reaction in living tissue. As used herein, “physiologically acceptable” is interchangeable with biocompatible.

As used herein, the term “hydrophobic” refers to a moiety or a molecule that is not attracted to water with significant apolar surface area at physiological pH and/or salt conditions. This phase separation can be observed via a combination of dynamic light scattering and aqueous NMR measurements. A hydrophobic therapeutic agent has a log P value of 1 or greater.

As used herein, the term “hydrophilic” refers to a moiety or a molecule that is attracted to and tends to be dissolved by water. The hydrophilic moiety is miscible with an aqueous phase. A hydrophilic therapeutic agent has a log P value of less than 1.

The log P values of hydrophobic and hydrophilic drugs can be found, for example, at pubchem.ncbi.nlm.nih.gov and drugbank.ca.

As used herein, the log P value is a constant defined in the following manner:

Log P=log 10 (Partition Coefficient)

Partition Coefficient, P=[organic]/[aqueous]

where [ ] indicates the concentration of solute in the organic and aqueous partition. A negative value for log P means the compound has a higher affinity for the aqueous phase (it is more hydrophilic); when log P=0 the compound is equally partitioned between the lipid and aqueous phases; a positive value for log P denotes a higher concentration in the lipid phase (i.e., the compound is more lipophilic). Log P=1 means there is a 10:1 partitioning in organic: aqueous phases. The most commonly used lipid and aqueous system is octan-1-ol and water, or octanol and buffer at a pH of 6.5 to 8.5.

As used herein, the term “water-insoluble” refers to a compound that has a water-solubility of less than 0.2 mg/mL (e.g., less than 0.1 mg/mL, or less than 0.01 mg/mL)), at a temperature of 25° C., and at a pressure of 1 atm or 101.3 kPa.

As used herein, the term “water-soluble” refers to a compound that is soluble in water in an amount of 1 mg/ml or more (e.g., 2 mg/ml or more), at a temperature of 25° C., and at a pressure of 1 atm or 101.3 kPa.

As used herein, the term “cationic” refers to a moiety that is positively charged, or ionizable to a positively charged moiety under physiological conditions. Examples of cationic moieties include, for example, amino, ammonium, pyridinium, imino, sulfonium, quaternary phosphonium groups, etc.

As used herein, the term “anionic” refers to a functional group that is negatively charged, or ionizable to a negatively charged moiety under physiological conditions. Examples of anionic groups include carboxylate, sulfate, sulfonate, phosphate, etc.

As used herein, the term “polymer” refers to a macromolecule having more than 10 repeating units.

As used herein, the term “small molecule” refers to a low molecular weight (<2000 Daltons) organic compound that may help regulate a biological process, with a size on the order of 1 nm. Most drugs are small molecules.

A number of antiviral therapeutic agents are referred to herein. Their names, molecular formula, molecular weight, water solubility, and structures are provided below.

Dolutegravir (DTV or D); also known as 1051375-16-6; GSK1349572; and Tivicay. Molecular formula: C₂₀H₁₉F₂N₃O₅. Molecular weight: 429.385 g/mol. Water solubility of <0.095 mg/mL; log P=2.2. IUPAC Name: (4R,9aS)-5-hydroxy-2-methyl-6,10-dioxo-3,4,6,9,9a,10-hexahydro-2H-1-oxa-4a,8a-diaza-anthracene-7-carboxylic acid-2,4 difluorobenzylamide. Chemical structure:

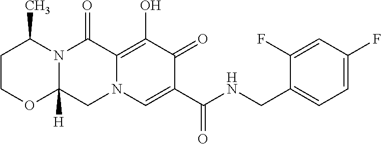 [SMILES: C[C@@H]1CCO[C@H]2Cn3cc(C(=O)NCc4ccc(F)cc4F)c(=O)c(O)c3C(=O)N12]

Lamivudine (3TC or L); also known as 134678-17-4; Epivir; Zeffix; Heptovir; and Epivir-HBV. Molecular formula: C₈H₁₁N₃O₃S. Molecular weight: 229.254 g/mol. Water solubility of 70 mg/mL; log P=−1.4. IUPAC Name: 4-amino-1-[(2R,5S)-2-(hydroxymethyl)-1,3-oxathiolan-5-yl]pyrimidin-2-one. Chemical structure:

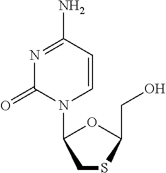

Tenofovir (TFV or T); also known as 147127-20-6; PMPA; Apropovir; (R)-9-(2-Phosphonomethoxypropyl)adenine; and D,L-Tenofovir. Molecular formula: C₉H₁₄N₅O₄P. Molecular weight: 287.216 g/mol. Water solubility of 13 mg/mL; log P=−1.6. IUPAC Name: [(2˜{R})-1-(6-aminopurin-9-yl)propan-2-yl]oxymethylphosphonic acid. Chemical structure:

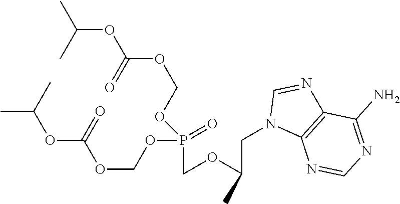 [SMILES: CC(C)OC(=O)OCOP(=O)(CO[C@H](C)Cn1cnc2c(N)ncnc21)OCOC(=O)OC(C)C]

Lopinavir (LPV); also known as ABT-378; 192725-17-0; Aluvia; Kaletra; and ABT 378. Molecular formula: C₃₇H₄₈N₄O₅. Molecular weight: 628.814 g/mol. Water solubility of 7.7×10⁻⁶ mg/mL; log P=5.94. IUPAC Name: (2S)—N-[(2S,4S,5S)-5-[[2-(2,6-dimethylphenoxy)acetyl]amino]-4-hydroxy-1,6-diphenylhexan-2-yl]-3-methyl-2-(2-oxo-1,3-diazinan-1-yl)butanamide. Chemical structure:

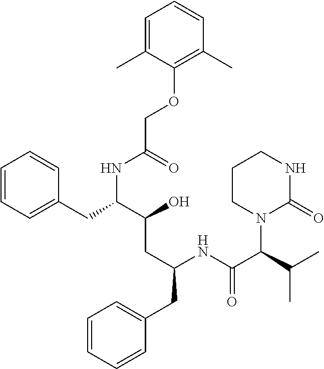 [SMILES: Cc1cccc(C)c1OCC(=O)N[C@@H](Cc1ccccc1)[C@@H](O)C[C@H](Cc1ccccc1)NC(=O)[C@H](C(C)C)N1CCCNC1=O]

Ritonavir (RTV); also known as 155213-67-5; Norvir; ABT-538; A-84538; and Abbott 84538. Molecular formula: C₃₇H₄₈N₆O₅S₂. Molecular weight: 720.948 g/mol. Water solubility of 1.1×10⁻⁷ mg/mL; log P=3.9. IUPAC Name: 1,3-thiazol-5-ylmethyl N-[(2S,3S,5S)-3-hydroxy-5-[[(2S)-3-methyl-2-[[methyl-[(2-propan-2-yl-1,3-thiazol-4-yl)methyl] carbamoyl]amino]butanoyl]amino]-1,6-diphenylhexan-2-yl]carbamate. Chemical structure:

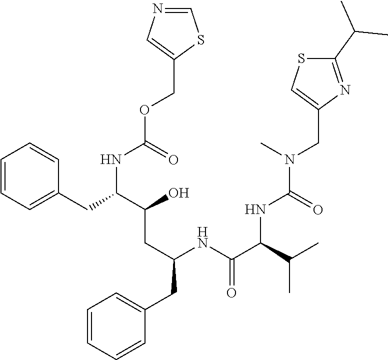 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

Rilpivirine (RPV), also known as Rilpivirine HCl, R278474 TMC 278: TMC-278:TMC278; and Edurant. Molecular formula: C₂₂H₁₉ClN₆. Molecular weight: 366.4 g/mol. Water solubility of 9.4×10−5 mg/mL; log P=4.5. IUPAC Name: 4-[[4-[4-[(E)-2-cyanoethenyl]-2,6-dimethylanilino]pyrimidin-2-yl]amino]benzonitrile. Chemical structure:

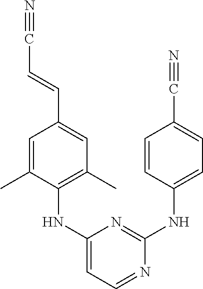 [SMILES: Cc1cc(/C=C/C#N)cc(C)c1Nc1ccnc(Nc2ccc(C#N)cc2)n1]

Efavirenz (EFV); also known as L 743,726; DMP226; Sustiva; Stocrin. Molecular Formula: C₁₄H₉ClF₃NO₂. Molecular weight: 628.814 g/mol. Water solubility of 8.55×10⁻⁶ mg/mL; log P=4. IUPAC Name: ((4S)-6-chloro-4-(2-cyclopropylethynyl)-4-(trifluoromethyl)-1H-3,1-benzoxazin-2-one. Chemical structure:

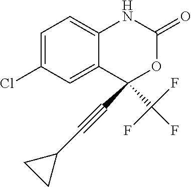 [SMILES: O=C1Nc2ccc(Cl)cc2[C@@](C#CC2CC2)(C(F)(F)F)O1]

As used herein, “absorption profile” refers to the rate and extent of exposure of a drug/combination of drugs, data analysis of the AUC and/or Cₘₐₓ including the curves thereof.

As used herein, “freely solubilized individual therapeutic agent” or “free soluble therapeutic agent” refers to a single therapeutic agent, or a salt thereof, fully dissolved in a pharmaceutically acceptable solvent such as saline, a buffer, or dimethyl sulfoxide (DMSO) (for experimental studies but not approved for formulating injectable as a solvent), without excipients such as a lipid and/or a lipid conjugate.

As used herein, “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, and transdermal. “Administering” can also include prescribing or filling a prescription for a dosage form comprising a particular compound/combination of compounds, as well as providing directions to carry out a method involving a particular compound/combination of compounds or a dosage form comprising the compound/combination of compounds.

As used herein, a “composition” refers to a collection of materials containing the specified components. One or more dosage forms may constitute a composition, so long as those dosage forms are associated and designed for use together.

As used herein, a “pharmaceutical composition” refers to a formulation of a compound/combination of compounds of the disclosure, and a medium generally accepted in the art for the delivery of the biologically active compound to mammals, e.g., humans. Such a medium includes all pharmaceutically acceptable carriers, diluents or excipients therefor. The pharmaceutical composition may be in various dosage forms or contain one or more unit-dose formulations. The pharmaceutical composition can provide stability over the useful life of the composition, for example, for a period of several months. The period of stability can vary depending on the intended use of the composition.

As used herein, “salts” include derivatives of an active agent, wherein the active agent is modified by making acid or base addition salts thereof. Examples of pharmaceutically acceptable salts include, but are not limited to, mineral or organic acid addition salts of basic residues such as amines; alkali or organic addition salts of acidic residues; and the like, or a combination comprising one or more of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, or a combination comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; or a combination comprising one or more of the foregoing salts.

As used herein, a “solid dispersion” relates to a solid system comprising a nearly homogeneous or homogeneous dispersion of an active ingredient/combination of active ingredients, in an inert carrier or matrix.

As used herein, a “homogeneous mixture” or “homogeneous distribution” refers to a mixture in which the components (e.g., APIs and excipients) are uniformly distributed throughout the mixture, which can be, for example, a suspension, a powder, or a solution. The mixture can have the same physical properties at every macroscopic sampling point of the assembled drug combination product.

As used herein, an “aqueous dispersion” refers to an aqueous suspension where the APIs and excipients of the pharmaceutical composition are suspended in a solvent or a buffer

“Prodrug” refers to a precursor of the pharmaceutically active agent wherein the precursor itself may or may not be pharmaceutically active but, upon administration, will be converted, either metabolically or otherwise, into the active agent or drug of interest. For example, prodrug includes an ester or an ether form of an active agent.

Particular pharmacokinetic parameters are defined in Table 1.

TABLE 1

| Parameter | Definition |
|---|---|
| AUC₀₋ₜ last | Area under the plasma concentration-time curve from |
| | time zero up to the last quantifiable concentration |
| AUC_{0-∞} | Area under the plasma concentration-time curve from |
| | time zero to infinity |
| % AUCₑₓₜᵣₐₚ | Percentage of AUC that is due to extrapolation from |
| | t last to infinity |
| Cₘₐₓ | Maximum observed plasma concentration |
| tₘₐₓ | Time of the maximum observed plasma concentration |
| t_{lag} | Time before the start of absorption |
| tₗₐₛₜ | Time of the last quantifiable plasma concentration |
| t_{1/2} | Apparent plasma terminal elimination half-life |
| | (terminal half-life) |

It is noted that AUC₀₋ₜ and AUC₀₋ₜₗₐₛₜ are used interchangeably herein. Also, AUC_{inf} and AUC_{t-inf} are used interchangeably with AUC_{0-∞}. It should also be understood that, unless otherwise specified, all pharmacokinetic parameters are measured after a single administration of the specified amount of a therapeutic agent/combination of therapeutic agents followed by a washout period in which no additional therapeutic agent/combination of therapeutic agents is administered.

A “terminal half-life” refers to the time required to divide the plasma concentration by two after reaching pseudo-equilibrium, and not the time required to eliminate half the administered dose. This is typically referred to as the last phase of descending plasma drug concentration over time and just before the drug is eliminated from the body.

A “therapeutically effective plasma concentration” refers to a plasma concentration of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

As an example, a therapeutically effective plasma concentration (EC₅₀) for Dolutegravir is about 0.02-2.14 nM, for Lamivudine is about 60 nM, for Tenofovir is about 0.04-8.5 μM, for Lopinavir is about 10-27 nM, for Ritonavir is about 3.8-153 nM, for Rilpivirine is about 0.7-1.1 nM, and for Efavirenz is about 1.7-25 nM.

As used herein, the phrase “therapeutically effective amount” refers to the amount of a therapeutic agent (i.e., drug, or therapeutic agent composition) that elicits the biological or medicinal response that is being sought in a tissue, system, animal, individual or human by a researcher, veterinarian, medical doctor or other clinician, which includes one or more of the following:
(1) preventing the disease; for example, preventing a disease, condition or disorder in an individual who may be predisposed to the disease, condition or disorder but does not yet experience or display the pathology or symptomatology of the disease;
(2) inhibiting the disease; for example, inhibiting a disease, condition or disorder in an individual who is experiencing or displaying the pathology or symptomatology of the disease, condition or disorder; and
(3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

As used herein, the term “composite” refers to a composition material, a material made from two or more constituent materials with significantly different physical or chemical properties that, when combined, produce a material with characteristics different from the individual components. The individual components remain separate and distinct within the finished structure.

As used herein, the term “individual,” “subject,” or “patient,” used interchangeably, refers to any animal, including mammals, preferably mice, rats, other rodents, rabbits, dogs, cats, swine, cattle, sheep, horses, or primates, and most preferably humans.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable sub-combination.

Furthermore, the particular arrangements shown in the FIGURES should not be viewed as limiting. It should be understood that other embodiments may include more or less of each element shown in a given FIGURE. Further, some of the illustrated elements may be combined or omitted. Yet further, an example embodiment may include elements that are not illustrated in the FIGURES.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Antiviral Therapeutic Agent Formulations

Powder Antiviral Therapeutic Agent Compositions

The antiviral therapeutic agent compositions of the present disclosure can form a homogeneous powder (e.g., a lyophilized homogeneous powder) having a homogeneous distribution of each antiviral therapeutic agent when viewed by scanning electron microscopy, such that each individual component is not visually discernible at 10-20 kV. The antiviral therapeutic agent compositions have a unified repetitive multi-drug motif (MDM) structure (used interchangeably herein with “multi-drug-lipid motif” and “multi-drug motif”), such that, unlike amorphous powders, the antiviral therapeutic agent compositions of the present disclosure have long range order, in the form of repetitive multi-drug and unified motifs. These motifs are homogenous or evenly distributed throughout the powder at any sampling point as determined by X-ray diffraction analysis, which can discern the physical organization of the drug combination structure stabilized by compatibilizer(s), which are homogenously distributed among the different therapeutic agent molecules.

The antiviral therapeutic agent compositions (which, as discussed above, can be in the form of a powder) can be made by fully dissolving water-insoluble antiviral agents and one or more compatibilizers in an alcoholic solvent, dissolving water-soluble antiviral agents in water or a water-based aqueous buffer; adding the buffer solution to the alcoholic solution to provide a mixture (e.g., a fully solubilized homogenous therapeutic agent and compatibilizer together in solution state), followed by a controlled removal of solvent in a process (e.g., a defined and controlled process) that locks the therapeutic agent and excipients into a unique powder product free of solvent and that has multi-drug motifs (MDM) with long range translational periodicity. These motifs are structurally different from purely amorphous material as verified by powder x-ray diffraction, and the antiviral therapeutic agent compositions can be hydrated and homogenized to produce long-acting injectable aqueous dispersions (e.g., in the form of a suspension) with 2-4 antiviral therapeutic agents, having a stability in suspension when stored for over 12 months at 4° C. The percentage of drug associated to the drug-combination particles is reproducible, and the particles are physically and chemically stable; thus, suitable for pharmaceutical preparation of long-acting injectable dosage form. The stable antiviral therapeutic agent compositions can provide long-acting therapeutic combinations having extended plasma antiviral therapeutic agent concentrations for the antiviral therapeutic agent components, compared to separately administered individual free antiviral therapeutic agent components, or an amorphous mixture of the antiviral therapeutic agents and excipients.

The antiviral therapeutic agent compositions can have a powder X-ray diffraction pattern that has at least one peak having a signal to noise ratio of greater than 3 (e.g., greater than 4, greater than 5, or greater than 6). The at least one peak can have a different 2θ peak position than the diffraction peak 2θ positions of each individual component (e.g., each individual therapeutic agent, or each individual therapeutic agent and excipient) of the antiviral therapeutic agent compositions. The at least one peak can have a different 2θ peak position than the diffraction peak 2θ positions for a simple physical mixture of the individual components of the antiviral therapeutic agent compositions. The X-ray diffraction pattern of the antiviral therapeutic agent compositions are indicative of multiple antiviral therapeutic agents assembled into a unified domain having repeating identical units, such that the antiviral therapeutic agents and the one or more compatibilizers together form an organized composition (as seen by the discrete powder X-ray diffraction peaks, described above). The organized composition can have a long-range order in the form of a repeating pattern organized as one unified structure, distinctly different from each X-ray diffraction profile for the drugs and lipid excipients. As used herein, short range order involves length scales of from 1 Å (or 0.1 nm) to 10 Å (or 1 nm), while long-range order has length scales that exceed 10 nm, or of an order that is at 2 theta 10-25 nm. The long-range order can be a characteristic feature of molecular spacing for a given molecule. Thus, the antiviral therapeutic agent compositions of the present disclosure have a unified repetitive multi-drug motif (MDM) structure and is referred to interchangeably herein as an “MDM composition.” MDM structures are described, for example, in Yu et al., J Pharm Sci 2020 November; 109(11):3480-3489, incorporated herein by reference in its entirety.

In some embodiments, the present disclosure features antiviral therapeutic agent compositions that include a combination of three or more antiviral therapeutic agents selected from dolutegravir (DTG), efavirenz (EFV), lopinavir (LPV), ritonavir (RTV), lamuvidine (3TC or L), abacavir, tenofovir (TFV) and prodrugs thereof (e.g., tenofovir disoproxil fumarate (TDF), tenofovir alafenamide (TAF)), emtricitabine (FTC), integrase inhibitors (raltigravir, elvitegravir, bictegravir, and cabotegravir), protease inhibitors (atazanavir (ATV), dauranavir, fosamprenavir, tipranavir), nucleoside reverse transcription inhibitors such as doravirine, non-nucleoside reverse transcription inhibitors such as MK-8591 (4′-ethynyl-2-fluoro-2′-deoxyadenosine), and rilpivirine. The antiviral therapeutic agent compositions include a mixture of water-soluble and water-insoluble antiviral therapeutic agents.

In some embodiments, a given antiviral therapeutic agent composition includes 1 or 2 water-insoluble therapeutic agents such as dolutegravir (DTG), efavirenz (EFV), lopinavir (LPV), ritonavir (RTV), and/or atazanavir (ATV), and 1 or 2 water-soluble therapeutic agents such as lamuvidine (3TC or L), abacavir, tenofovir (TFV) and prodrugs thereof (e.g., tenofovir disoproxil fumarate (TDF), tenofovir alafenamide (TAF)), and/or emtricitabine (FTC)), and the antiviral therapeutic agent composition can include a mixture of 3 or 4 antiviral therapeutic agents.

For example, the antiviral therapeutic agent composition can include a combination of three or more therapeutic agents such as: a combination of dolutegravir, lamuvidine, and tenofovir and prodrugs thereof; a combination of lopinavir, ritonavir, and tenofovir and prodrugs thereof; a combination of efavirenz, lopinavir, and tenofovir and prodrugs thereof; a combination of atazanavir, ritonavir, and tenofovir and prodrugs thereof; a combination of lopinavir, ritonavir, lamuvidine, tenofovir and prodrugs thereof; a combination of efavirenz, tenofovir disoproxil fumarate, and emtricitabine (FTC); a combination of dolutegravir, tenofovir disoproxil fumarate, and emtricitabine; a combination of dolutegravir, lamuvidine, and tenofovir disoproxil fumarate; or a combination of dolutegravir, lamuvidine, and abacavir.

In some embodiments, the antiviral therapeutic agent composition includes a combination of dolutegravir, lamuvidine, and tenofovir and prodrugs thereof; a combination of efavirenz, lopinavir, and tenofovir and prodrugs thereof; a combination of lopinavir, ritonavir, lamuvidine, tenofovir and prodrugs thereof; a combination of efavirenz, tenofovir disoproxil fumarate, and emtricitabine (FTC); a combination of dolutegravir, tenofovir disoproxil fumarate, and emtricitabine; a combination of dolutegravir, lamuvidine, and tenofovir disoproxil fumarate; a combination of dolutegravir, lamuvidine, and abacavir; a combination of dolutegravir, lamuvidine, tenofovir and prodrugs thereof, and rilpivirine.

In some embodiments, the antiviral therapeutic agent composition includes a combination of atazanavir:ritonavir:tenofovir and prodrugs thereof at a molar ratio of about 2:1:3; a combination of lopinavir, ritonavir, and tenofovir and prodrugs thereof at a molar ratio of about 4:1:5; a combination of lopinavir, ritonavir, lamuvidine, and tenofovir and prodrugs thereof at a molar ratio of about 4:1:4:5; a combination of efavirenz, lopinavir, and tenofovir and prodrugs thereof at a molar ratio of about 0.8:1:15; or a combination of dolutegravir, lamuvidine, and tenofovir and prodrugs thereof has a molar ratio of about 1:1:1:0.5.

In some embodiments, the combination of antiviral therapeutic agents is efavirenz, lopinavir, and tenofovir and prodrugs thereof at a molar ratio of about 0.8:1:15. In some embodiments, the combination of antiviral therapeutic agents includes tenofovir and prodrugs thereof: lamuvidine:dolutegravir at a molar ratio of from about 15:15:15.3 to about 21:26.2:14.4. In some embodiments, the combination of antiviral therapeutic agents is lopinavir, ritonavir, lamuvidine, and tenofovir and prodrugs thereof at a molar ratio of about 4:1:4:5. In some embodiments, the combination of antiviral therapeutic agents is dolutegravir, lamuvidine, tenofovir and prodrugs thereof, and rilpivirine at a molar ratio of about 1:1:1:0.5.

In some embodiments, the combination of antiviral therapeutic agents in the antiviral therapeutic agent composition includes tenofovir and prodrugs thereof: lamuvidine:dolutegravir at a molar ratio of from about 1:1:1 (from about 2:2:1, from about 3:3:1, from about 4:4:1, or from about 5:5:1) to about 6:6:1 (e.g., to about 5:5:1, to about 4:4:1, to about 3:3:1, or to about 2:2:1). In some embodiments, the combination of antiviral therapeutic agents in the antiviral therapeutic agent composition includes tenofovir and prodrugs thereof: lamuvidine:dolutegravir at a molar ratio of from about 15:15:15.3 to about 21:26.2:14.4; from about 2:2:1 to about 6:6:1, from about 3:3:1 to about 6:6:1, from about 4:4:1 to about 6:6:1, from about 5:5:1 to about 6:6:1, from about 2:2:1 to about 5:5:1, from about 3:3:1 to about 5:5:1, from about 3:3:1 to about 4:4:1).

The antiviral therapeutic agent compositions can exhibit a therapeutically effective plasma concentration of the combination of antiviral therapeutic agents for 3 or more weeks (e.g., 4 or more weeks, 5 or more weeks, 6 or more weeks, 7 or more weeks, or 8 or more weeks).

In some embodiments, the antiviral therapeutic agent compositions of the present disclosure exhibit a therapeutically effective plasma concentration of the combination of antiviral therapeutic agents for 2 or more weeks (e.g., 3 or more weeks, 4 or more weeks 5 or more weeks, 6 or more weeks, 7 or more weeks, or 8 or more weeks), when administered to a subject in need thereof as a bolus dose.

The antiviral therapeutic agent compositions of the present disclosure further include one or more compatibilizers such as a lipid and/or a lipid conjugate, in addition to the combination of antiviral therapeutic agents. In some embodiments, the one or more compatibilizers is present in the antiviral therapeutic agent composition in an amount of 60 wt % or more (e.g., 70 wt % or more, 80 wt % or more, 90 wt % or more) and 95 wt % or less (e.g., 90 wt % or less, 80 wt % or less, or 70 wt % or less) relative to the weight of the total antiviral therapeutic agent composition. In some embodiments, the one or more compatibilizers, such as a covalent conjugate of a lipid with a hydrophilic moiety (e.g., PEG-DSPE, mPEG-DSPE, or mPEG₂₀₀₀-DSPE), is present in the antiviral therapeutic agent composition in an amount of 2 mole % or more (e.g., 5 mole % or more, 8 mole % or more, or 10 mole % or more) and 15 mole % or less (e.g., 10 mole % or less, 8 mole % or less, or 5 mole % or less) relative to the total compatibilizer content. In some embodiments, the one or more compatibilizers, such as a covalent conjugate of a lipid with a hydrophilic moiety (e.g., PEG-DSPE, mPEG-DSPE, or mPEG₂₀₀₀-DSPE), is present in the antiviral therapeutic agent composition in an amount of 10 mole % relative to the total compatibilizer content. In some embodiments, a covalent conjugate of a lipid with a hydrophilic moiety (e.g., PEG-DSPE, mPEG-DSPE, or mPEG₂₀₀₀-DSPE) in a mole percent of lower than 15% (e.g., 12%, or 10%) compared to the total compatibilizer content provides a composition exhibiting a sustained therapeutically effective plasma concentration of the constituent therapeutic agents over a period of at least 1 week (e.g., at least 2 weeks, at least 3 weeks, or at least 1 month), while a mole percent of greater than 15% (e.g., 20% or more) provides a therapeutically effective plasma concentration half-life of less than 2 days.

The one or more compatibilizers can include at least one lipid excipient and at least one lipid conjugate excipient. For example, the one or more compatibilizers can include at least one lipid excipient in an amount of 50 wt % or more and 80 wt % or less. The lipid excipient can be a saturated or unsaturated lipid excipient, such as a phospholipid. The phospholipid can include, for example, 1,2-distearoyl-sn-glycero-3-phosphocholine (DSPC), 1,2-dimyristoyl-sn-glycero-3-phosphocholine (DMPC), 1,2-dipalmitoyl-sn-glycero-3-phosphocholine (DPPC). In some embodiments, the one or more compatibilizers include at least one lipid conjugate excipient in an amount of 19 wt % or more and 25 wt % or less relative to the weight of the total antiviral therapeutic agent composition. The lipid conjugate excipient can be a covalent conjugate of a lipid with a hydrophilic moiety. The hydrophilic moiety can include a hydrophilic polymer, such as poly(ethylene glycol) having a molecular weight (Mₙ) of from 500 to 5000 (e.g., from 500 to 4000, from 500 to 3000, from 500 to 2000, from 1000 to 5000, from 1000 to 4000, from 1000 to 3000, from 1000 to 2000, from 2000 to 5000, from 2000 to 4000, from 2000 to 3000, 2000, 1000, 5000, or 500). In some embodiments, the lipid conjugate excipient is a conjugate of 1,2-distearoyl-sn-glycero-3-phosphoethanolamine (DSPE) with PEG, such as PEG₂₀₀₀ or mPEG₂₀₀₀ The PEG can be conjugated to the lipid via an amide linkage. The lipid conjugate excipient can be in the form of a salt, such as an ammonium or a sodium salt.

In some embodiments, the one or more compatibilizers is 1,2-distearoyl-sn-glycero-3-phosphocholine and/or 1,2-distearoyl-sn-glycero-3-phosphoethanolamine-N-[poly(ethylene glycol)2000]. In some embodiments, the compatibilizers in the antiviral therapeutic agent composition is 1,2-distearoyl-sn-glycero-3-phosphocholine and 1,2-distearoyl-sn-glycero-3-phosphoethanolamine-N-[poly(ethylene glycol)2000].

The antiviral therapeutic agent compositions in powder form can include the antiviral therapeutic agents and the one or more compatibilizers together in an organized composition. The antiviral therapeutic agents and the one or more compatibilizers together can have a long-range order in the form of a repeating pattern. The antiviral therapeutic agents and the one or more compatibilizers together can include a repetitive multi-drug motif (“MDM”) structure.

In some embodiments, the antiviral therapeutic agent compositions in powder form do not include a lipid layer excipient, a lipid bilayer excipient, a liposome, a micelle, or any combination thereof. In some embodiments, the antiviral therapeutic agent compositions are not amorphous (e.g., having a broad undefined X-ray diffraction pattern), but have discrete powder X-ray diffraction peaks indicative of organization and/or longrange order in the form of repeating patterns. In some embodiments, the antiviral therapeutic agent compositions are not in the form of an implant (e.g., a subdermal implant). In some embodiments, the antiviral therapeutic agent in the antiviral therapeutic agent composition is present in its native, salt, or solvate form, but a prodrug thereof is not required to provide the long-acting injectable aqueous dispersion. In some embodiments, the antiviral therapeutic agent compositions do not include nano/microcrystalline forms of the therapeutic agents or the compatibilizer(s).

In some embodiments, the antiviral therapeutic agent composition of the present disclosure is not an amorphous solid dispersion. Rather, a given antiviral therapeutic agent composition is not a physical mixture or a blend of its constituent antiviral therapeutic agents and excipients, and as such, possesses properties unique to the composition that are different from those of each of the constituent antiviral therapeutic agents and excipients. For example, the antiviral therapeutic agent compositions can have a phase transition temperature different from the transition temperature of each individual component when assessed by differential scanning calorimetry. In some embodiments, one or more of the transition temperatures of each individual component is no longer present in the antiviral therapeutic agent compositions, which include an organized assembly of the antiviral therapeutic agent and excipient components (i.e., one or more compatibilizers). In some embodiments, the antiviral therapeutic agent compositions have a homogeneous distribution of each individual therapeutic agent when viewed by scanning electron microscopy, such that each individual component is not visually discernible at 10-20 kV.

The antiviral therapeutic agent compositions can remain stable when stored at 25° C. for at least 2 weeks (e.g., at least 3 weeks, at least 4 weeks, at least 6 weeks, or at least 8 weeks) and/or up to 12 months (e.g., up to 6 months, up to 6 months, or up to 4 months), at a relative humidity of 20% to 80%, at a pressure of 1 atm, and in air (i.e., 21% oxygen and 78% nitrogen), such that the at least one X-ray diffraction peak at position(s) corresponding to a given antiviral therapeutic agent composition are preserved over the time period. In some embodiments, both the X-ray diffraction peak positions and intensities are preserved when the composition is stored at 25° C. for at least 2 weeks (e.g., at least 3 weeks, at least 4 weeks, at least 6 weeks, or at least 8 weeks) and/or up to 12 months (e.g., up to 6 months, up to 6 months, or up to 4 months).

In some embodiments, a given antiviral therapeutic agent composition includes each antiviral therapeutic agent in an amount of 2 wt % or more (e.g., 3 wt % or more, 5 wt % or more, 10 wt % or more, or 15 wt % or more) and 20 wt % or less (e.g., 15 wt % or less, 10 wt % or less, 5 wt % or less, or 3 wt % or less) relative to the weight of the total antiviral therapeutic agent composition.

The antiviral therapeutic agent compositions can include a molar ratio of the sum of antiviral therapeutic agents to the one or more compatibilizers of from 30:115 to 71:40 (e.g., from 40:115 to 71:40, from 50:100 to 71:40, from 60:100 to 71:40, from 70:100 to 71:40, from 70:90 to 71:50, from 70:80 to 71:50, or from 70:70 to 71:50). In certain embodiments, the antiviral therapeutic agent compositions can include a molar ratio of the sum of antiviral therapeutic agents to the one or more compatibilizers of from about 1:10 (e.g., from about 1:9, from about 1:8, from about 1:7, from about 1:6, from about 1:5, from about 1:4, from about 1:3, or from about 1:2) to about 1:1 (e.g., to about 1:2, to about 1:3, to about 1:4, to about 1:5, to about 1:6, to about 1.7, to about 1:8, or to about 1:9). In certain embodiments, the antiviral therapeutic agent compositions can include a molar ratio of the sum of antiviral therapeutic agents to the one or more compatibilizers of from about 1:7 to about 1:2.

The antiviral therapeutic agent compositions can be a solid. For example, the antiviral therapeutic agent compositions can be a powder. The powder can be formed of particles having an average dimension of from 100 nm (e.g., from 500 nm, from 1 μm, from 4 μm, from 6 μm, or from 8 μm) to 10 μm (e.g., to 8 μm, to 6 μm, to 4 μm, to 1 μm, or to 500 nm). The average dimension (e.g., a diameter) of a particle can be determined by transmission and/or scanning electron microscopy, averaged over 500 particles. In some embodiments, particle diameter can be measured using photon correlation spectroscopy.

Aqueous Dispersions

The present disclosure also features injectable aqueous dispersions including an aqueous solvent, and an antiviral therapeutic agent composition dispersed in the aqueous solvent to provide the injectable aqueous dispersion. The injectable aqueous dispersions exhibit a therapeutically effective plasma concentration of the combination of antiviral therapeutic agents for 2 or more weeks (from a single injected bolus dose).

The antiviral therapeutic agent composition can be in powder form prior to dispersion in the aqueous solvent to provide the aqueous dispersion. The powder form of the antiviral therapeutic agent composition is described above. The antiviral therapeutic agent composition powder can be mixed with an aqueous solvent to provide an aqueous dispersion. The aqueous dispersion can be a suspension of the antiviral therapeutic agent composition. In some embodiments, once suspended in the aqueous solvent, the size of the suspended particles of the antiviral therapeutic agent composition is reduced (e.g., to less than 0.2 μm) prior to administration to a subject, for example, by subjecting the aqueous dispersion to a homogenizer and/or a sonicator. The aqueous dispersion can then be optionally filtered to remove any microorganisms, for example, through a 0.2 μm filter. The aqueous dispersion is adapted to be parenterally administered to a subject. As used herein, parenteral administration refers to a medicine taken into the body or administered in a manner other than through the digestive tract, such as by intravenous or subcutaneous administration

The antiviral therapeutic agents in the antiviral therapeutic agent compositions can be present at various molar ratios. For example, the combination of antiviral therapeutic agents can include efavirenz, lopinavir, and tenofovir and prodrugs thereof at a molar ratio of about 0.8:1:15. As another example, the combination of antiviral therapeutic agents can include tenofovir and prodrugs thereof: lamuvidine:dolutegravir at a molar ratio of from about 1:1:1 (from about 2:2:1, from about 3:3:1, from about 4:4:1, or from about 5:5:1) to about 6:6:1 (e.g., to about 5:5:1, to about 4:4:1, to about 3:3:1, or to about 2:2:1). In some embodiments, the combination of antiviral therapeutic agents can include tenofovir and prodrugs thereof: lamuvidine:dolutegravir at a molar ratio of from about 15:15:15.3 to about 21:26.2:14.4; from about 2:2:1 to about 6:6:1, from about 3:3:1 to about 6:6:1, from about 4:4:1 to about 6:6:1, from about 5:5:1 to about 6:6:1, from about 2:2:1 to about 5:5:1, from about 3:3:1 to about 5:5:1, from about 3:3:1 to about 4:4:1). As yet another example, the combination of antiviral therapeutic agents can include lopinavir, ritonavir, lamuvidine, and tenofovir and prodrugs thereof at a molar ratio of about 4:1:4:5. In some embodiments, the combination of antiviral therapeutic agents includes dolutegravir, lamuvidine, tenofovir and prodrugs thereof, and rilpivirine at a molar ratio of about 1:1:1:0.5. The combination of antiviral therapeutic agents at these ratios can exhibit sustained plasma concentrations of 2 weeks or more, 3 weeks or more, 4 weeks or more, 5 weeks or more, or 6 weeks or more, from a single injected bolus dose. As used herein, a sustained plasma concentration is a plasma drug concentration that is maintained for a defined period (e.g., 14 days or more and/or 90 days or less) above the EC₅₀ value of each antiviral therapeutic agent in the combination of therapeutic agents, and at a dosage without adverse effects (e.g., pain and other untoward effects as defined in a clinical product label). The plasma drug concentration is determined from the blood taken from the subject over time and the drug levels determined with a validated assay in the plasma (separated from the coagulated blood and free of red cells).

In some embodiments, the injectable aqueous dispersions exhibit a therapeutically effective plasma concentration of the combination of antiviral therapeutic agents for 3 or more weeks, from a single injected dose. In some embodiments, the injectable aqueous dispersions exhibit a therapeutically effective plasma concentration of the combination of antiviral therapeutic agents for 4 or more weeks, from a single injected dose. In some embodiments, the injectable aqueous dispersions exhibit a therapeutically effective plasma concentration of the combination of antiviral therapeutic agents for 5 or more weeks, after a single injected dose. In some embodiments, the injectable aqueous dispersions exhibit a therapeutically effective plasma concentration of the combination of antiviral therapeutic agents for 6 or more weeks, after a single injected dose.

In the aqueous dispersion, the antiviral therapeutic agents and the one or more compatibilizers together can form an organized composition, as discussed above. In the aqueous dispersion, the antiviral therapeutic agents and the one or more compatibilizers together can have a long range order in the form of a repeating pattern. In the aqueous dispersion, the antiviral therapeutic agents and the one or more compatibilizers together can include a repetitive multi-drug motif (“MDM”) structure.

In some embodiments, the aqueous dispersions do not include a lipid layer excipient, a lipid bilayer excipient, a liposome, a micelle, or any combination thereof. The aqueous dispersions do not include an antiviral therapeutic agent composition that is amorphous. In some embodiments, the aqueous dispersions are not in the form of nor incorporated in an implant (e.g., a subdermal implant). In some embodiments, the antiviral therapeutic agent in the aqueous dispersions is present in its native, salt, or solvate form, but a prodrug thereof is not needed to provide the long-acting injectable aqueous dispersion. In some embodiments, the aqueous dispersions of the present disclosure do not include nano/microcrystalline forms of the therapeutic agents and/or the compatibilizer(s).

In some embodiments, the aqueous solvent is a buffered aqueous solvent, saline, or any balanced isotonic physiologically compatible buffer suitable for administration to a subject, as known to a person of skill in the art. For example, the aqueous solvent can be an aqueous solution of 20 mM sodium bicarbonate and 0.45 wt % to 0.9 wt % NaCl.

A given aqueous dispersion can include the antiviral therapeutic agent composition in an amount of 10 wt % or more (e.g., 15 wt % or more, or 20 wt % or more) and 25 wt % or less (e.g., 20 wt % or less, or 15 wt % or less), relative to the final aqueous dispersion.

The aqueous dispersions of the antiviral therapeutic agent composition of the present disclosure can provide a therapeutically effective plasma concentration of the antiviral therapeutic agents over a longer period of time compared an aqueous dispersion of a physical mixture of the antiviral therapeutic agents and excipients, an amorphous mixture of the therapeutic agents and excipients, or compared to separately administered antiviral therapeutic agents at a same dosage. In some embodiments, the aqueous dispersions of the antiviral therapeutic agent composition provide from 2 (e.g., from 5, from 10, or from 15) to 50 (e.g., to 40, to 30, or to 20) fold higher exposure (e.g., AUC_{0-24 h} calculated from plasma drug concentrations using the trapezoidal rule) of each antiviral therapeutic agent in the antiviral therapeutic agent composition in non-human primates, when administered parenterally (e.g., subcutaneously), when compared to non-human primates treated with an equivalent dose of the same free and soluble therapeutic agent individually in solution. In some embodiments, the aqueous dispersions of the antiviral therapeutic agent composition provide from 20-fold (e.g., from 30 fold, or from 40 fold) to 50 fold (e.g., to 40 fold, or to 30 fold) higher exposure (e.g., AUC_{0-24 h} calculated from plasma drug concentrations using the trapezoidal rule) of each antiviral therapeutic agent in the antiviral therapeutic agent composition in non-human primates, when administered parenterally (e.g., subcutaneously), when compared to non-human primates treated with an equivalent dose of the same free and soluble antiviral therapeutic agent individually in solution.

In some embodiments, the aqueous dispersions of the antiviral therapeutic agent compositions of the present disclosure are long-acting, such that the parenteral administration of the aqueous dispersion can occur once every 2 weeks (e.g., every 3 weeks, every 4 weeks, or every 5 weeks) to once every 6 weeks (e.g., every 5 weeks, every 4 weeks, or every 3 weeks).

In certain embodiments, the aqueous dispersions of the antiviral therapeutic agent compositions of the present disclosure have a terminal half-life greater than the terminal half-life of each freely solubilized individual antiviral therapeutic agent. For example, the antiviral therapeutic agent compositions and aqueous dispersions thereof can have a half-life extension of greater than 2 to 3-fold of each constituent antiviral therapeutic agent's individual elimination half-life. In some embodiments, the antiviral therapeutic agent compositions and aqueous dispersions thereof can have a half-life extension of from 8-fold (e.g., from 10-fold, from 15-fold, from 20-fold, from 30-fold, from 40-fold, or from 50-fold) to 62-fold (e.g., to 50-fold, to 40-fold, to 30-fold, to 20-fold, to 15-fold, or to 10-fold) for each constituent therapeutic agent's individual elimination half-life.

The particles of antiviral therapeutic agent compositions in the aqueous dispersion can maintain the MDM organization of the antiviral therapeutic agents and the one or more compatibilizers, such that the physically-assembled stable molecular organization of the therapeutic agents and the compatibilizer is preserved. In some embodiments, the particles of the antiviral therapeutic agent composition in the aqueous dispersion do not form a lipid layer, a lipid bilayer, a liposome, or a micelle in the aqueous solvent. In some embodiments, the particles of the antiviral therapeutic agent composition in the aqueous dispersion do not include a nanocrystalline antiviral therapeutic agent. In some embodiments, after hydration of the antiviral therapeutic agent composition, the particles of antiviral therapeutic agent compositions are discoidal rather than spherical, when visualized by transmission electron microscopy. For example, the discoid particles of the antiviral therapeutic agent compositions, after suspension in an aqueous solvent, can have a dimension of, for example, a width of from 5 nm (e.g., from 8 nm, from 10 nm, or from nm) to 20 nm (e.g., to 15 nm, to 10 nm, or to 8 nm) by a length of from 30 nm (e.g., from 35 nm, from 40 nm, or from 45 nm) to 50 nm (e.g., to 45 nm, to 40 nm, or to 35 nm), having a thickness of from 3 nm (e.g., from 5 nm, from 7 nm) to 10 nm (e.g., to 7 nm, to 5 nm), as visualized by transmission electron microscopy.

The particles of the antiviral therapeutic agent composition in the aqueous dispersion can have a maximum dimension of from 10 nm (e.g., 25 nm, 50 nm, 100 nm, 150 nm, 200 nm) to 300 nm (e.g., 200 nm, 150 nm, 100 nm, 50 nm, or 25 nm). Particle diameter can be measured using photon correlation spectroscopy.

As used herein, the “aqueous dispersion” refers to a suspension of the antiviral therapeutic agent composition in the aqueous solvent, where the antiviral therapeutic agent composition is present in the form of insoluble particles suspended, stably in the aqueous solvent. In some embodiments, rather than an aqueous dispersion, the antiviral therapeutic agent composition can be dissolved in an aqueous solvent to provide a solution. When the antiviral therapeutic agent composition is in a solution, it is solubilized and dissolved in the solvent.

### Methods of Treatment

The present invention provides a method of prevention, treatment or prophylaxis of diseases caused by retroviruses, especially acquired immune deficiency syndrome or an HIV infection, including administering the injectable aqueous dispersion including the antiviral therapeutic agent composition as described herein.

The present disclosure features methods of treating diseases caused by retroviruses, including parenterally administering to a subject in need thereof, at a frequency of at most one dose every 2 weeks, any of the injectable aqueous dispersion as described above. The subject can be HIV-positive. The dose can be a bolus dose. As used herein, “parenteral administration” refers to a medicine taken into the body or administered in a manner other than through the digestive tract, such as by intravenous or subcutaneous administration. In some embodiments, parenteral administration does not include intramuscular administration.

For example, the methods can include parenterally administering to a subject in need thereof, at a frequency of at most one dose every 2 weeks an aqueous dispersion including an aqueous solvent, and an antiviral therapeutic agent composition dispersed in the aqueous solvent.

As discussed above, in some embodiments, the antiviral therapeutic agent composition include a combination of antiviral therapeutic agents, such as a combination of dolutegravir, lamuvidine, and tenofovir and prodrugs thereof; a combination of efavirenz, lopinavir, and tenofovir and prodrugs thereof; a combination of lopinavir, ritonavir, lamuvidine, tenofovir and prodrugs thereof; a combination of efavirenz, tenofovir disoproxil fumarate, and emtricitabine (FTC); a combination of dolutegravir, tenofovir disoproxil fumarate, and emtricitabine; a combination of dolutegravir, lamuvidine, and tenofovir disoproxil fumarate; a combination of dolutegravir, lamuvidine, and abacavir; a combination of dolutegravir, lamuvidine, tenofovir and prodrugs thereof, and rilpivirine. The antiviral therapeutic agent compositions further include one or more compatibilizers including a lipid, a lipid conjugate, or a combination thereof.

In some embodiments, the combination of antiviral therapeutic agents is efavirenz, lopinavir, and tenofovir and prodrugs thereof at a molar ratio of about 0.8:1:15. In some embodiments, the combination of antiviral therapeutic agents is tenofovir and prodrugs thereof: lamuvidine:dolutegravir at a molar ratio of from about 15:15:15.3 to about 21:26.2:14.4. In some embodiments, the combination of antiviral therapeutic agents is lopinavir, ritonavir, lamuvidine, and tenofovir and prodrugs thereof at a molar ratio of about 4:1:4:5. In certain embodiments, the combination of antiviral therapeutic agents is dolutegravir, lamuvidine, tenofovir and prodrugs thereof, and rilpivirine at a molar ratio of about 1:1:1:0.5.

In some embodiments, parenteral administration of the aqueous dispersion to the subject occurs at a frequency of at most one dose per every 3 week (e.g., ATV:RTV:TFV at a molar ratio of 2:1:3; LPV:RTV:TFV at a molar ratio of 4:1:5; or EFV:LPV:TFV at a ratio of 0.8:1:1.5). In some embodiments, parenteral administration of the aqueous dispersion to the subject occurs at a frequency of at most one dose per every 4 weeks (e.g., tenofovir and prodrugs thereof: lamuvidine:dolutegravir at a molar ratio of from about 15:15:15.3 to about 21:26.2:14.4; or lopinavir, ritonavir, lamuvidine, and tenofovir and prodrugs thereof at a molar ratio of about 4:1:4:5; or dolutegravir, lamuvidine, tenofovir and prodrugs thereof, and rilpivirine at a molar ratio of about 1:1:1:0.5). In some embodiments, parenteral administration of the aqueous dispersion to the subject occurs at a frequency of at most one dose per every 5 weeks. In some embodiments, parenteral administration of the aqueous dispersion to the subject occurs at a frequency of at most one dose per every 6 weeks.

In some embodiments, parenteral administration of the aqueous dispersion to the subject occurs at a frequency of one dose per every 3 week, 4 weeks, 5 weeks, 6 weeks, 7 weeks, or 8 weeks, or a combination thereof.

In some embodiments, the aqueous dispersion is administered intravenously. In some embodiments, the aqueous dispersion is administered subcutaneously. In some embodiments, the aqueous dispersion is not administered intramuscularly.

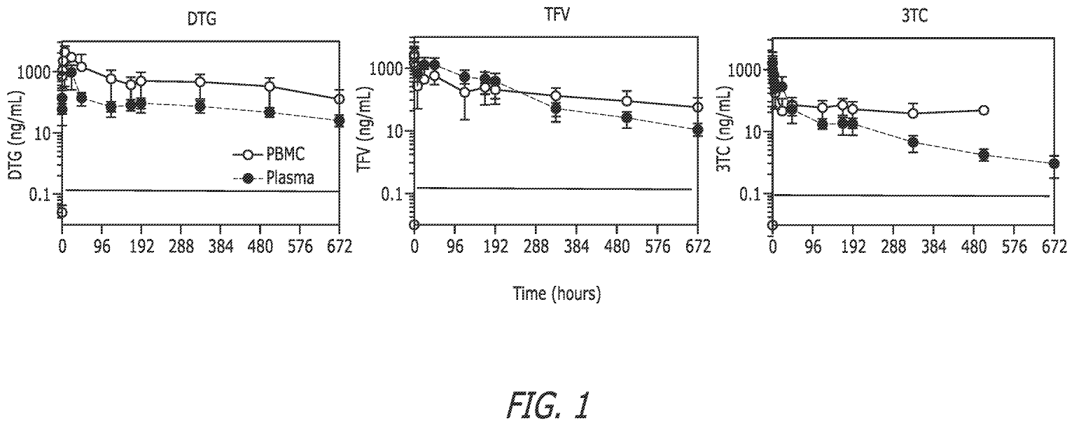

Unlike sustained release liposome and polymeric formulations deposited at injection sites which peak slowly and may take days to reach therapeutic drug levels, the antiviral therapeutic agent compositions and the aqueous dispersions of the present disclosure can make a fraction (e.g., from 5 to 10%) of the therapeutic agents available soon within hours after administration to provide a loading-dose-like behavior (see, FIG. 1), thereby making an oral or IV companion dose unnecessary for treating HIV patients with such long-acting drug combination products.

### Methods of Making the Aqueous Dispersions

General Procedure

The process of making an injectable aqueous dispersion including an antiviral therapeutic agent composition that includes water-soluble and water-insoluble antiviral therapeutic agents (to provide long-acting pharmacokinetic characteristics) can generally be performed in three steps.

Step 1—Production of the Antiviral Therapeutic Agent Composition in Powder Form

1 or 2 therapeutic agents from the water insoluble category, such as LPV and RTV or DTG in solid states, can first be dissolved together with one or more compatibilizers (e.g., DSPC and mPEG₂₀₀₀-DPSE) in a container with alcoholic solvent at a temperature 60-70° C. Then water-soluble drugs such as TDF, TFV, TAF, 3TC, FTC (e.g., at a concentration of about 10 to 50 mg/ml) were prepared in buffered aqueous solution at pH 5-8 (e.g., a 0.45 (w/v) % NaCl buffered aqueous solution) at 60-70° C. Then the water-soluble drugs in buffered solution are added drop-wise into water insoluble drugs which are fully dissolved in ethanol at 60-70° C. such that the final total solid concentration in the ethanol-water (9:1 v/v) solution is 5-10 (w/v) %. When all component-drugs and lipids are in solution, the mixture can be spray-dried (e.g., with Procept M8TriX (Zelzate, Belgium) or Buchi B290). For example, for Procept instruments, inlet temperature for the spray dryer can be maintained at 70° C. with an inlet air speed of 0.3 m³/min and chamber pressure of mBar. Dried drug combination nanoparticle powder generated by the spray-dryer can be collected; and subjected to vacuum desiccation. The dried powder antiviral therapeutic agent composition can be characterized with powder X-ray diffraction to be free of individual drug crystal signatures, but with a cohesive unified X-ray diffraction pattern representing multiple drug (combination) domains (MDM) assembled in repeating units. The MDM diffraction pattern can be different from that of amorphous X-ray diffraction presented typically as a broad halo with no single peak in the drug powder products. In addition, in contrast to a metastable state of amorphous organization that return to individual drug x-ray signatures of crystalline form, the single unified peak in the X-ray diffraction for the antiviral therapeutic agent composition powder, which was contributed by MDM ordering, can be stable at 25-30° C. for months (e.g., more than 6 months, more than 9 months, more than 12 months).

Step 2—Production of the Aqueous Dispersion

The powder antiviral therapeutic agent composition can be resuspended in buffer (e.g., 0.45 NaCl containing 50 mM NaHCO₃, pH 7.5) at 65-70° C. to provide an aqueous suspension. After the powder is in suspension, the mixture can be allowed to hydrate (absorbing water to DcNP powder containing MDM structure) with mixing at elevated temperatures (e.g., 65-70° C. for 2-4 hours, pH 7-8). The suspension can be subjected to size reduction (e.g., with a homogenizer until a uniform particle size between 10 nm and 300 nm mean diameter). Particle diameter can be measured using photon correlation spectroscopy.

Step 3—Sterile Injectable Aqueous Dispersion

To produce a sterile injectable suspension, the suspension can be sterilized using methods known to a skilled practitioner. For example, the step 2 process can be performed either under aseptic conditions in a class II biosafety sterile cabinet or the aqueous dispersion can be filtered through 0.2 μm terminal sterilization filter. The final injectable aqueous dispersion can be collected in a sterile glass vial; sterility can be verified by exposing the product on a blood agar plate test for 7 days with no bacterial growth.

### Bioanalytical Assays to Determine Therapeutic Agent Concentration in Plasma and Cells

Plasma therapeutic agent concentrations can be measured using an assay developed and validated previously (see, e.g., Kraft et al., J Control Release. 2018 Apr. 10; 275: 229-241, incorporated herein by reference in its entirety). The lower limit of quantification can be 0.01 nM for the therapeutic agents in plasma.

Effects of the Injectable Aqueous Dispersion on Antiviral Drug Combinations on Long-Acting Plasma and Cellular Kinetics in Non-Human Primates

Subjects (e.g., macaques) can be subcutaneously administered with either antiviral therapeutic agent aqueous dispersion or soluble free drug combination. The free-drug combination control groups include administering an indicated single subcutaneous dose where equivalent dug combinations are dissolved in DMSO and diluted with water to the subjects.

Venous blood samples can be collected from a femoral vein at predetermined days and times after subcutaneous injection. Whole blood in EDTA tubes can be immediately centrifuged and plasma can be removed and frozen at −80° C. until LC-MS/MS analysis.

Plasma drug concentrations can be in units of nM. Non-compartmental parameters can be estimated from plasma profiles for free and DcNP formulations using Phoenix WinNonlin (Certara, Princeton, N.J.). The following non-compartmental parameters can be estimated: area under the plasma concentration-time curve (AUC) extrapolated to infinity; terminal half-life (t½); apparent clearance (CL/F); and mean body residence time (MBRT) based on moments extrapolated to infinity.

Evaluation of Peripheral Blood Mononuclear Cells (PBMC) and Lymph Node Mononuclear Cells (LNMC)

PBMCs can be isolated from whole blood using density gradient centrifugation and divided into pellets of 2×10⁶ cells each. Lymph nodes, surgically excised at indicated time points after drug administration, can be dissociated by pressing the tissue through a 100 μm nylon cell strainer (Corning; Tewksbury, Mass.). They can be suspended in cell culture media, followed by similar gradient sedimentation treatment as that of PBMC to isolate lymph node mononuclear cells (LNMCs), and can then be analyzed for drug concentrations based on 2×10⁶ cells for each sample/time point. All samples can be stored at −80° C. prior to LC-MS/MS drug analysis.

Intracellular concentrations of each drug in the injectable aqueous dispersion can initially be calculated as pg/million cells. For comparison to plasma extracellular drug concentrations, PBMC intracellular concentrations can be converted to nM based on an average mononuclear cell volume of 4×10⁻⁹ mL.

The Examples below describe compositions and processes to physically transform 2 or more antiviral therapeutic agents of disparate properties into long-acting injectable aqueous dispersions.

### Examples

General Procedures

An approach integrating composition and process to physically transform 2 or more drugs of disparate properties into a long-acting medicinal drug combination product is described. Several drug-combinations were used to verify this integrated approach for making long-acting medicine. The first step was to make a drug combination powder composed of 2 or more drugs with disparate properties (water-solubility characteristics). The resulting unique powder was distinct from typical amorphous drug products as the drug-combination nanoparticle (DcNP) composition exhibits unique, unified, and uniform collective patterns detectable via X-ray diffraction. When the drug-combination DcNP powder product was suspended in a buffer, followed by size reduction, it formed a stable nano-sized drug-combination suspension suitable as an injectable dosage. This approach was used successfully to prepare more than 5 sets of HIV drug combinations, including a combination of world-wide interest called TLD (TDF or tenofovir (T); Lamivudine (L) (also known as 3TC); Dolutegravir (D)). Primate data indicated that this technology was surprisingly successful useful and enabled the transformation of daily oral short-acting TLD into a long-acting form that lasts 4 weeks in non-human primates (NHP). This technology could be used to make long-acting drug combinations including from 2 to 4 drugs in one dosage.

Materials

All HIV drugs used were pharmaceutical grade and manufactured under current good manufacturing processes (cGMP) that met specifications of purity and quality. The test compounds or active pharmaceutical ingredients (APIs) used in this study could be sorted into two generally categories according to their water solubility. Water insoluble HIV drugs included Dolutegravir (DTG), Efavirenz (EFV), Lopinavir (LPV), Ritonavir (RTV), Atazanavir (ATV); water-soluble HIV drugs included Lamivudine (3TC or L), Tenofovir (TFV) and its prodrugs Tenofovir disoproxil fumarate (TDF) and Tenofovir alafenamide (TAF), Emtricitabine (FTC). cGMP lipid excipients-1,2-distearoyl-sn-glycero-3-phosphocholine (DSPC) and 1,2-distearoyl-sn-glycero-3-phosphoethanolamine-N-[poly (ethylene glycol)2000] (mPEG₂₀₀₀-DSPE) were purchased from Cordon Pharma (Liestal, Switzerland). Anhydrous ethanol was purchased from Decon Pharmaceuticals (King of Prussia, Pa.). Other reagents and salts were of high purity, analytical or pharmaceutical grade or higher quality.

Preparation of Drug Combination Nanoparticle (DcNP) Injectable Dosage Form

The process of making an injectable drug combination including water soluble and insoluble drugs (intended to provide long-acting pharmacokinetic characteristics) was generally performed in three key steps. They are as follows:

Step 1—Production of Drug Combination Particles in Powder Form

Typically, one or two drugs from the water insoluble category, such as LPV and RTV or DTG in solid states, were first dissolved together with DSPC and mPEG₂₀₀₀-DPSE in a glass container with ethanol at 60-70° C. Then 10-50 mg/ml of water-soluble drugs such as TDF, TFV, TAF, 3TC, FTC were prepared in a 0.45 (w/v) % NaCl buffered solution (pH 5-8) at 60-70° C. Then the water-soluble drugs in buffered solution were added drop wise into water insoluble drugs dissolved in ethanol at 60-70° C. such that the final total solid concentration in the ethanol-water (9:1 v/v) solution was 5-10 (w/v) %. When all component-drugs and lipids were solubilized, the mixture was spray-dried either using a Procept M8TriX (Zelzate, Belgium) or Buchi B290. For Procept instruments, inlet temperature for the spray dryer was maintained at 70° C. with an inlet air speed of 0.3 m³/min and chamber pressure of 25 mBar. Dried drug combination nanoparticle powder generated by the spray-dryer was collected; and subjected to vacuum desiccation for 48 hr. The dried powder DcNP products were characterized with powder X-ray diffraction to be free of individual drug crystal signatures but provided a cohesive unified X-ray diffraction pattern representing multiple drug (combination) domains (MDM) assembled in repeating units. The MDM diffraction pattern was also different from that of amorphous X-ray diffraction presented typically as a broad halo with no single peak in the drug powder products. In addition, in contrast to a metastable state of amorphous organization that typically returned to individual drug x-ray signatures of crystalline form, the single unified peak observed in X-ray diffraction for DcNP powder, which was contributed by MDM ordering, was stable at 25-30° C. for more than 6 months.

Step 2—DcNP Suspension and Particle Size Reduction

The powder DcNP composed of 2 lipid excipients and hydrophobic water insoluble drugs, such as DTG or LPV and RTV plus hydrophilic water soluble TFV or 3TC or both, were resuspended in 0.45 NaCl containing 50 mM NaHCO₃, pH 7.5 at 65-70° C. After all the drugs in the DcNP powder were in suspension, the mixture was allowed to hydrate with gentle mixing at 65-70° C. for 2-4 hours (hr), pH 7-8. Then the suspension was subjected to size reduction with a homogenizer (Avestin Emulsiflex 5, Ottawa, Ontario, Canada; Microfluidics LM20, Westwood, Mass.) in a continuous cycle operating at 8-20 k psi until a uniform particle size between 50-100 nm mean diameter and 98% less than 200 nm based on photon correlation spectroscopy (Nicomp 380 PCS, Santa Barbara, Calif.).

Step 3—Sterile Injectable DcNP Dosage Form

To produce a sterile injectable suspension, the step 2 process was performed either under aseptic conditions in a class II biosafety sterile cabinet or by filtration through 0.2 polycarbonate filter. The final injectable product was collected into a sterile glass vial; sterility was verified by exposing the product on a blood agar plate test for 7 days with no bacterial growth. The sterile materials were used for NHP studies.

Analytical Methods

Powder X-Ray Diffraction

Powder X-ray Diffraction (PXRD) was performed on a Bruker D8 Focus X-ray Diffractor (Madison, Wis., USA) with Cu-Kα radiation. Operational voltage and amperage were set to 40.0 kV and 40.0 mA, respectively. XRD profile scan parameters included a step size of 0.035° 2θ in an operating range of 5° to 50° 2θ. Powder (˜100-200 mg) was pressed into a sample container to obtain a flat upper surface. The two lipid excipients-1,2-distearoyl-sn-glycero-3-phosphocholine (DSPC) and 1,2-distearoyl-sn-glycero-3-phosphoethanolamine-N-[poly (ethylene glycol)2000] (mPEG₂₀₀₀-DSPE); as well as all the active pharmaceutical ingredients or drugs—Dolutegravir (DTG), Efavirenz (EFV), Lopinavir (LPV), Ritonavir (RTV), Atazanavir (ATV); Lamivudine (3TC or L), Tenofovir (TFV) and its prodrug Tenofovir disoproxil fumarate (TDF) and Tenofovir alafenamide (TAF), Emtricitabine (FTC)-all exhibited unique identifiable crystalline peak characteristic each on XRD scan profile over the 2-50 degree 20.

Evaluation of Peripheral Blood Mononuclear Cells (PBMC) and Lymph Node Mononuclear Cells (LNMC)

PBMCs were isolated from whole blood using density gradient centrifugation and divided into pellets of 2×10⁶ cells each. Lymph nodes, surgically excised at indicated time points after drug administration, were dissociated by pressing the tissue through a 100 μm nylon cell strainer (Corning; Tewksbury, Mass.). They were suspended in cell culture media, followed by similar gradient sedimentation treatment as that of PBMC to isolate lymph node mononuclear cells (LNMCs), and were then analyzed for drug concentrations based on 2×10⁶ cells for each sample/time point. All samples were stored at −80° C. prior to LC-MS/MS drug analysis.

Bioanalytical Assay to Determine Drugs in Plasma and Cells

Plasma drug concentrations were measured using an assay developed and validated previously (see, e.g., Kraft et al., J Control Release. 2018 Apr. 10; 275: 229-241, incorporated herein by reference in its entirety). The lower limit of quantification was 0.01 nM for all three drugs in plasma.

For determination of drug concentrations in PBMC and LNMC, pellets of 2×10⁶ cells/tube were lysed using 200 μL water/methanol (50:50 v/v). To ensure complete lysis, the samples were sonicated for 10 min Subsequent extraction and analysis was the same as for plasma. The lower limit of quantification was 0.01 nM for lysed cell suspension concentration converted.

Effects of DcNP Formulation on HIV Drug Combinations on Long-Acting Plasma and Cellular Kinetics in Non-Human Primates

To evaluate whether presenting the three drugs—e.g., DTG-3TC-TFV, LPV-RTV-TFV, EFV-LPV-TFV, ATZ-RTV-TFV or 4 drugs—e.g., LPV-RTV-3TC-TFV in a combination nanosuspension using DcNP platform could provide long-acting plasma and intracellular drug exposure, macaques were subcutaneously administered with either DcNP formulated or soluble free drug combination. The free-drug combination (control) arm consisted of two groups. In the control group, 2-4 macaques were administered at an indicated single subcutaneous dose where equivalent dug combinations are dissolved in DMSO and diluted with water.

Venous blood samples were collected from a femoral vein at 0, 0.5, 1, 3, 5, 8, 24, 48, 120, 168, 192 and 336 hours (14 days), 21 and 28 days after subcutaneous injection. Whole blood in EDTA tubes was immediately centrifuged and plasma was removed and frozen at −80° C. until LC-MS/MS analysis.

Plasma drug concentrations were reported in units of nM. Non-compartmental parameters were estimated from plasma profiles for free and DcNP formulations using Phoenix WinNonlin (Certara, Princeton, N.J.). The following non-compartmental parameters were estimated: area under the plasma concentration-time curve (AUC) extrapolated to infinity; terminal half-life (t_{1/2}); apparent clearance (CL/F); and mean body residence time (MBRT) based on moments extrapolated to infinity.

Intracellular concentrations of each drug in the DcNP were initially calculated as pg/million cells. For comparison to plasma extracellular drug concentrations, PBMC intracellular concentrations were converted to nM based on an average mononuclear cell volume of 4×10⁻⁹ mL.

### Example 1: Transformation of Short-Acting Current 3-Oral-Drug Combination TLD, or Tenofovir (T or TFV)-Lamivudine (L or 3TC)-Dolutegravir-(D or DTG), into an Injectable Drug Combination Nanoparticulate Dosage Form that Exhibit Long-Acting Pharmacokinetics

Characteristics of the Drug Combination Nanoparticle (DcNP) Dosage Formulation that Enable Small Drug-Combination Particles in Suspension that Meet Injectable Dosage Form

To evaluate whether the water-soluble TFV and 3TC (Log P<1) could be combined with water-insoluble DTG (Log P >2), the 3 drugs were fully dissolved, together with the two lipid excipients in hydrated hot ethanol (˜5% v/v) or other co-solvent, followed by controlled solvent removal by spray drying or lyophilization (step 1, described above). The resulting DcNP power was kept under vacuum to remove residual water. Then the DcNP powder was resuspended in buffered saline at pH 7-8 at 60-70° C. After being fully hydrated, the DcNP suspension was subjected to size reduction by sonication, homogenization, extrusion or microfluidization (step 2, described above). Typical injectable final dosage form product was in 30-250 nm diameter and less than 1 μm, and stable as injectable suspension.

This process was reproducible and the results of multiple preparations of TLD injectable sterile DcNP in suspension were as follows (Tables 1-3).

TABLE 1
Step 1-Preparation and characterization of stable
DcNP powder (Step 1) and quality verified by XRD.

| | | | Step 1- | Powder |
|---|---|---|---|---|
| | | | solvent | quality Per |
| TFV:3TC:DTG | | % | removal | XRD |
| (mole ratio) | Solvent composition | solid | method | (Pass/Fail) |
| 15:15:15.3 | CHCl₃:EtOH:H₂O | 1.9 | Rotor | Pass |
| | (65:35:4 v/v/v) | | Evaporation | |
| 15:15:15.3 | CHCl₃:EtOH:H₂O | 2.8 | Rotor | Pass |
| | (65:35:4 v/v/v) | | Evaporation | |
| 21:26.2:14.4 | EtOH:H₂0 (90:10 v/v) | 10 | Spray Dry | Pass |
| 21:26.2:14.4 | EtOH:H₂0 (90:10 v/v) | 5 | Spray Dry | Pass |

Inference and summary: As shown in Table 1, two different ratios of TLD drug composition were dissolved in co-solvent —CHCl₃:EtOH:H₂O (65:35:4 v/v/v) or ethanol:water or buffer (EtOH:H₂O 95:5 v/v) were evaluated with two different methods of controlled solvent removal. After rotor-evaporation of spray drying removed solvent at defined conditions to control the rate of solvent removal, the formation of stable DcNP powder could be verified by X-ray diffraction pattern of the powder. Instead of formation of halo or broad scan across X ray angle, which reflect the amorphous powder product or crystalline peaks for each drug in the mixture, the DcNP powder exhibited a unique and unified peak for all drugs in the mixture plus the two lipid excipients. The quality and formation of the DcNP powder product enabled by this step 1 process was graded as pass or fail (P/F) in Table 1. The powder produced without addition of lipid excipients or solvent removal process was not under controlled conditions. When the drugs and lipids were not fully dissolved in the solvent prior to controlled solvent removal, the produced powder generally failed the XRD quality test.

Various lipid excipients either in addition to or substituting the two lipid excipients, including addition of up to 30 mole percent of cholesterol and derivatives; varying lengths (and MW) of PEG of DSPE-PEG2000; or varying the fatty acyl chain degree of saturation, chain lengths of phosphatidyl choline, as well as modification could be done to produce DcNP powder.

TABLE 2
Step 2- DcNP suspension and particle size reduction
and verification by formation of stable nanoparticles
in suspension and degree of drug association.

| | | Particle | % DcNP association | | |
|---|---|---|---|---|---|
| TFV:3TC:DTG | Size reduction | size (mean | (under sink condition) | | |
| (mole ratio) | Method | in nm) | TFV | 3TC | DTG |
| 15:15:15.3 | Sonication | 34.6 | 16.3 | 20.3 | 69.6 |
| 15:15:15.3 | Sonication | 31.2 | 26.0 | 32.0 | 86.0 |
| 21:26.2:14.4 | Homogenization | 35.6 | 21.9 | 13.4 | 89.2 |
| 21:26.2:14.4 | Microfluidization | 31.2 | 10.6 | 4.9 | 85.7 |

Inference and summary: As shown in Table 2, after hydration of the DcNP powder in buffered saline for approximately 1-4 hr, followed by size reduction, the final particle size of DcNP in suspension could be made to meet the USP criteria for injectable products. Regardless of the size reduction methods—sonication to homogenization or microfluidization (readily could be scaled up for making commercial products)—the final DcNP product in suspension exhibited particle diameters less than 200 nm. Most (>95%) are within 100 nm.

Under sink conditions (i.e., removing drug by dialyzing the product against a large volume of buffer), significant fractions of the 3 drugs formulated in DcNP remained associated for 4 hrs. While not all drugs formulated in DcNP remained associated, even water-soluble drugs (i.e., TFV and 3TC) remained associated to DcNP particles in suspension. This was surprising. These % DcNP association data could be reproduced for each prescribed composition and method listed above; thus, the DcNP in suspension did not require the removal of unassociated drugs for use as an injectable dosage form. The fraction of the drug presented as DcNP unassociated could allow for achieving an early peak in plasma drug concentrations after dosing—which was typically referred to as a loading dose.

Unlike drugs formulated in other sustained release liposome and polymeric formulations which, when deposited at injection sites, can take days to reach therapeutic drug levels in plasma after injection (referred to as slow rise to target drug concentrations) and therefore require an IV or oral lead-in or loading dose to reach therapeutic drug level more rapidly and within the therapeutic time frame, the DcNP renders a fraction of the drug available soon after administration. Therefore, drugs in DcNP dosage form provide a loading-dose-like behavior, making an oral or IV companion dose unnecessary for treating HIV patients with all-in-one strategy within such long-acting drug combination products.

TABLE 3
(Step 3) Sterile injectable DcNP dosage form.

| | | % recovery after 0.2 μm terminal | | |
|---|---|---|---|---|
| | | sterile filtration | | |
| TFV:3TC:DTG | Particle size | (under sink condition) | | |
| (mole ratio) | (mean in nm) | TFV | 3TC | DTG |
| 21:26.2:14.4 | 35.6 | >99 | >98 | >98 |
| 21:26.2:14.4 | 31.2 | >99 | >98 | >98 |

To evaluate its use as an injectable dosage form, the DcNP suspension was required to meet the standards as prescribed in the USP with respect to sterility and freedom from microbial content. Initial preparations were made under the aseptic processing conditions from step 2 with hydration and size reduction under a validated sterile hood. The aseptically prepared DcNP suspension was tested to be free from microbes based on a 14-day blood agar-microbial test and validated endotoxin assay. The DcNP suspension was stable in 4° C. storage for more than 6 months. As the mean particle size was less than 200 nm and met the upper limit of large particle counts for injectable dosage forms.

To enable non-aseptic processing where terminal filtration through 0.2 μm filter may be required, the feasibility of this terminal sterilization by 0.2 μm filter approach was evaluated.

Inference and summary: The results summarized in Table 3 indicated that 0.2 μm terminal filtration was feasible as validated by % of the 3 drugs, TFV, 3TC and DTG recovered after filtration. The filtration materials were evaluated and generally polycarbonate as well as PES (hydrophilic polyethersulfone) could be used to produce a similar degree of recovery. As hydrophilic polyethersulfone or PES polymers were typically used to prepare sterile injection products—and found in pharmaceutical sterilization and in terminal filter sterilization products under brand names such as Supor EKV—the use of terminal sterile filtration of DcNP suspension for preparation of sterile dosage form was feasible.

Evaluation of DcNP Containing TLD 3-Drug Injectable Dosage Form for Long-Acting Characteristics in Primates

To evaluate the ability of the DcNP injectable suspension product with characteristics mentioned above, a batch of DcNP containing tenofovir (TFV or T, lamivudine (3TC or L) and dolutegravir (DTG or D) was given as a single subcutaneous dose to primates. Blood and cell (PBMC) samples were collected from these primates over 4 weeks. The concentration in blood and PBMC (cells) for each drug given in DcNP particles as a single injectable suspension was determined with an LC-MS/MS mass-spectrometric assay and presented in FIG. 1 as plasma time-concentration profile.

Inference and summary: The results summarized in FIG. 1 clearly demonstrate that a single dose of the 3 current oral HIV drugs, commonly used as a first line daily oral drug-combination for treatment, when given to primates in a DcNP formulated injection, produced long-lasting plasma drug levels for the 4 week study in primates. Thus, daily dosing could be converted into dosing once every 4-weeks. It is believed that giving primates a higher dose could provide even longer duration of drugs in plasma—it is likely that less frequent dosing could be realized with DcNP formulated TLD. As a reference, orally administered TLD exhibited a plasma half-life around 6-8 hours and cleared within 24 hours. Also, since oral tablet/transit time in the human gastro-intestinal (GI) tract was 8-24 hours, they were unlikely to remain in the system for more than a day or two. Thus, DcNP technology enabled the transformation of a short-acting daily dosing regimen into a long-acting every 4-week dosing for the same 3 drugs-TLD.

| | Oral single dose | DcNP formulation |
|---|---|---|
| | formulation | (non-human primates) |
| | (plasma half- | (plasma half- |
| Therapeutic agent | life, hours) | life, hours) |
| Lopinavir | 2.5 | >250-350 |
| Tenofovir (TDF/TAF) | 8-17 | >200-350 |
| Dolutegravir (DTG) | 23 | >400 |
| Ritonavir | 2.5 | >250-350 |
| 3TC (Lamivudine) | 6 | >100-200 |

Key attributes of the DcNP transformative technology which enabled repurposing oral short-acting drug combinations to long-acting dosage formulation were as follows: (1) technical readiness and novel processes and compositions to transform current standard of care drugs to long-acting formulations with validation in NHPs (2) the DcNP all-in-one injectable form without a need for cold chain, simplifying adherence and implementation (3) an innovative and accelerated FDA regulatory pathway in place (4) proven demonstration of LA pharmacokinetics for all active drugs of interest (5) a simplified 2-step manufacturing process that could be adapted for commercial scale production and implementation in LMICs (6) added benefits of prolonged drug exposure in cells and tissues to potentially accelerate HIV clearance.

### Example 2. General Application of 3-4 Antiviral Drugs and Different Compositions to Validate DcNP Enabling Technology

To evaluate whether the DcNP technology was generally applicable to transform current HIV drugs, a number of drug combinations composed of commonly prescribed HIV drugs for daily or more frequent dosing (or oral cART) were selected. More than 5 different drug combinations were evaluated for formulation stability and suitability for scaling as well as stability. 4 sets of drug combinations, including one 4 HIV-drug combination in DcNP dosage form, were further evaluated in primates. One of the combinations has been tested in rats and dogs to further demonstrate long-acting pharmacokinetic across species. These data are summarized in the following Table 4.

Table 4. DcNP platform technology enable transformation of short-acting HIV drugs into once-a week or more long-acting dosage form—Additional compositions evaluated.

| | ATV plus | 101 | 102 | EFV plus | 301 plus |
|---|---|---|---|---|---|
| DcNP | (3 API) | (3API) | (4 API) | (3 APi) | (4 API) |
| Drug composition | ATV:RTV:TFV | LPV:RTV:TFV | LPV:RTV:3TC:TFV | EFV:LPV:TFV | DTG:3TC:TFV:RPV |
| Drug mole ratio | 2:1:3 | 4:1:5 | 4:1:4:5 | 0.8:1:1.5 | 1:1:1:0.5 |
| Primate dose | 25; 12.8; 15 | 25:7.1:10.6 | 25:7:10.6:10.6 | 25:16.7:22.6 | |
| (mg/kg each) | | | | | |
| Formulation stable | Yes | Yes | Yes | Yes | Yes |
| and scalable as | | | | | |
| injectable dosage | | | | | |
| form | | | | | |
| Long acting in | Yes | Yes | Yes | Yes | Yes |
| NHP | | | | | |
| Duration of Drug | 2 weeks for | 2 weeks for | 5 weeks for all | 2 weeks for all | 4 weeks or |
| persistence in | all drugs in | all drugs in | drugs in | drugs in plasma | more in |
| plasma | plasma and | plasma and | plasma; 5 | and cells | plasma |
| | cells | cells | weeks in cells | | |
| Cell:Plasma | >1 all drugs | >1 all drugs | >1 all drugs | >1 all drugs | >1 all drugs |
| drug ratios | | | | | |
| Safety in NHP | Basic | Single and | Basic | Basic | Basic |
| | | 6-month q2w | | | |
| Animal species | Primates | Primates | Primates | Primates | Primates |
| validated | | Rat-single | | | |
| | | Rat- qw 5x | | | |
| | | Dog-single | | | |

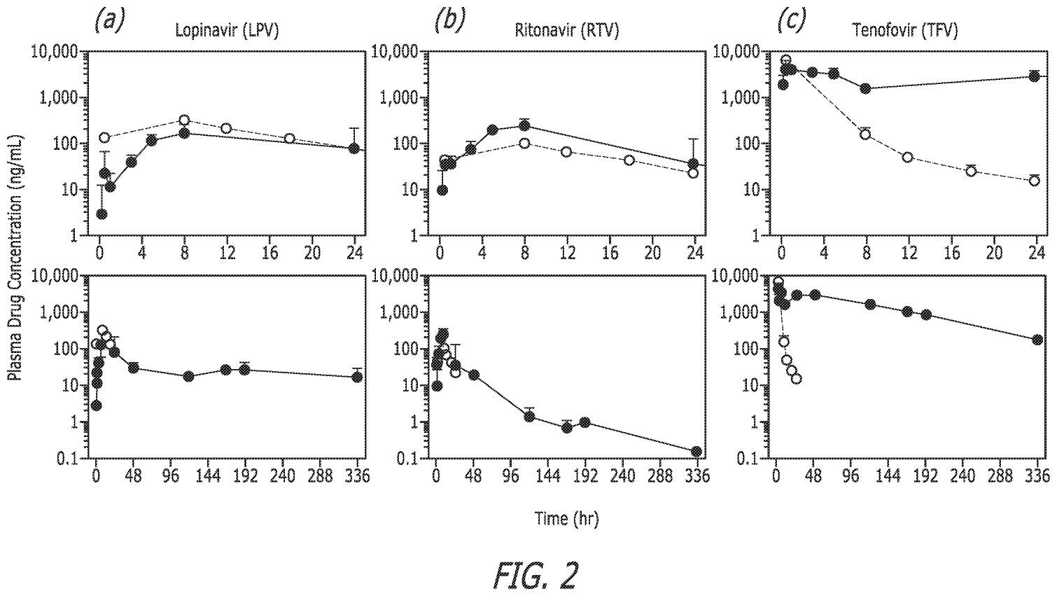

FIG. 2 is a graph showing plasma concentration-time profiles of lopinavir, ritonavir, and tenofovir in macaques following a single subcutaneous dose of the antiviral therapeutic agents in either the free, soluble therapeutic agent (open circles and dotted lines) or in an injectable aqueous dispersion of the present disclosure (closed circles and solid line). The top graphs in panels (a), (b), and (c) show the plasma concentration-time profiles of the first 24 hours after subcutaneous dosing, and the bottom graphs are the entire time course over 336 hours (2 weeks). Plasma limit of quantification (LOQ)/limit of detection (LOD)=lopinavir: 10/4, ritonavir: 50/25, tenofovir: 250/100 pg/mL, ritonavir (intended as a PK booster for lopinavir) plasma after dosing of the injectable aqueous dispersion was <LOQ in N=2 at 192 and 336 hours. Geometric mean+/−SD (N=3-8). No detectable therapeutic agent was seen at about 24 hours for all 3 free therapeutic agents when given together (as free therapeutic agents (open circle). In contrast, when the same 3 drugs were given in DcNP dosage form (closed circle), all three drugs were detectable over at least the 2-week study period, demonstrating long-acting kinetic property of DcNP dosage form.

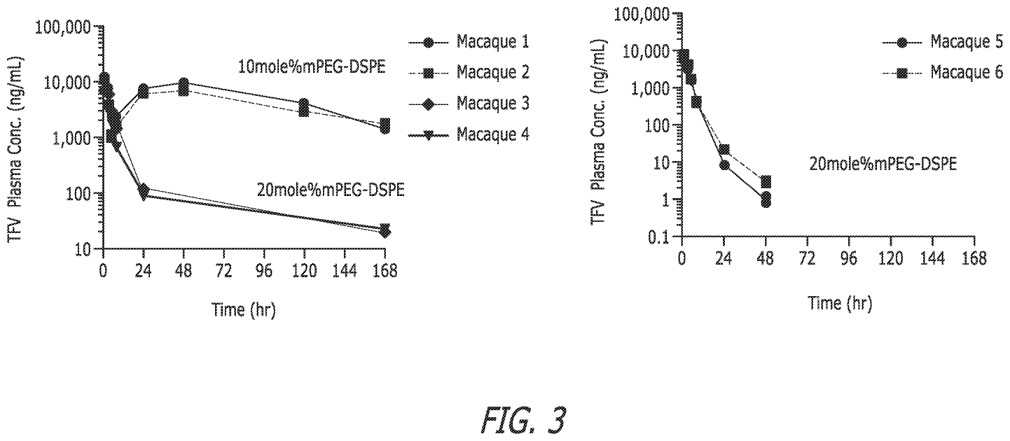

FIG. 3 is a series of graphs showing the effect of the composition of compatibilizers on the plasma concentration over time of a therapeutic agent, when administered as an injectable aqueous dispersion of the present disclosure (as part of a DcNP combination of 3 therapeutic agents LPV/RTV/TFV and mPEG₂₀₀₀-DSPE+DSPC compatibilizer). Monitoring the TFV plasma concentration, at 10 mole % mPEG₂₀₀₀-DSPE in the total compatibilizer, the combination of therapeutic agents of the present disclosure provided sustained release in plasma (when administered to macaques) over at least 168 hours, whereas compositions including 20 mole % mPEG₂₀₀₀-DSPE in the total compatibilizer had little plasma concentration after 48 hours. LPV and RTV followed the same trendlines. Each line of the graphs represents one macaque monkey, administered subcutaneously with the LPV/RTV/TFV and varying mPEG₂₀₀₀-DSPE DcNP composition.

Inference and summary: The results summarized in Table 4 demonstrated that 5 formulations with different sets of drugs in DcNP combination could be made that meet injectable dosage form. Table 4 also listed the specific and diverse ratio of drug compositions that could be used in producing the injectable dosage form. These specific dosage forms could also scale and meet the injectable dosage form criteria for testing long-acting pharmacokinetics in primates, and other rodent and non-rodent higher animal species to provide safety and confirm long acting pharmacokinetics.

Collectively, these data demonstrate the utility of DcNP technology to produce a diverse set of drug combinations. Up to 4 drugs with different hydrophobicity and hydrophilicity could be combined into one injectable suspension. When the DcNP drug combination in suspension was injected into animals, the resultant product could transform short-acting, daily oral dosing into long-acting, weekly or less frequent dosing regimens.

Thus, this DcNP technology could transform short-acting drugs (with disparate physical properties) into long-acting forms. These long-acting drug-combination dosage forms can be used to improve patient adherence as chronic daily dosing often leads to poor patient compliance from pill fatigue. Adherence was necessary to provide sustained therapeutic effects, particularly to sustain HIV suppression to prevent patients from progressing into AIDS and death.

By example and without limitation, embodiments are disclosed according to the following enumerated paragraphs:
A1. An injectable aqueous dispersion, comprising:
an aqueous solvent, and
an antiviral therapeutic agent composition dispersed in the aqueous solvent to provide the injectable aqueous dispersion, the antiviral therapeutic agent composition comprising a combination of antiviral therapeutic agents selected from:
   dolutegravir, lamuvidine, and tenofovir and prodrugs thereof;
   efavirenz, lopinavir, and tenofovir and prodrugs thereof;
   lopinavir, ritonavir, lamuvidine, tenofovir and prodrugs thereof;
   efavirenz, tenofovir disoproxil fumarate, and emtricitabine (FTC);
   dolutegravir, tenofovir disoproxil fumarate, and emtricitabine;
   dolutegravir, lamuvidine, and tenofovir disoproxil fumarate;
   dolutegravir, lamuvidine, and abacavir;
   dolutegravir, lamuvidine, tenofovir and prodrugs thereof, and rilpivirine;
   and
the antiviral therapeutic agent composition further comprising one or more compatibilizers comprising a lipid, a lipid conjugate, or a combination thereof;
wherein the injectable aqueous dispersion exhibits a therapeutically effective plasma concentration of the combination of antiviral therapeutic agents for 2 or more weeks.
A2. The aqueous dispersion of Paragraph A1, wherein the one or more compatibilizers are selected from 1,2-distearoyl-sn-glycero-3-phosphocholine, 1,2-distearoyl-sn-glycero-3-phosphoethanolamine-N-[poly(ethylene glycol)2000], and a combination thereof.
A3. The aqueous dispersion of Paragraph A1 or Paragraph A2, wherein the combination of antiviral therapeutic agents is efavirenz, lopinavir, and tenofovir and prodrugs thereof at a molar ratio of about 0.8:1:15.
A4. The aqueous dispersion of Paragraph A1 or Paragraph A2, wherein the combination of antiviral therapeutic agents comprises tenofovir and prodrugs thereof: lamuvidine:dolutegravir at a molar ratio of from about 15:15:15.3 to about 21:26.2:14.4.
A5. The aqueous dispersion of Paragraph A1 or Paragraph A2, wherein the combination of antiviral therapeutic agents is selected from:
lopinavir, ritonavir, lamuvidine, and tenofovir and prodrugs thereof at a molar ratio of about 4:1:4:5; and
dolutegravir, lamuvidine, tenofovir and prodrugs thereof, and rilpivirine at a molar ratio of about 1:1:1:0.5.
A6. The aqueous dispersion of Paragraph A4 or Paragraph A5, wherein the injectable aqueous dispersion exhibits a therapeutically effective plasma concentration of the combination of antiviral therapeutic agents for 3 or more weeks.
A7. The aqueous dispersion of Paragraph A4 or Paragraph A5, wherein the injectable aqueous dispersion exhibits a therapeutically effective plasma concentration of the combination of antiviral therapeutic agents for 4 or more weeks.
A8. The aqueous dispersion of any one of Paragraphs A1 to A7, wherein the antiviral therapeutic agents and the one or more compatibilizers together form an organized composition.
A9. The aqueous dispersion of any one of Paragraphs A1 to A8, wherein the antiviral therapeutic agents and the one or more compatibilizers together comprise a long-range order in the form of a repeating pattern.
A10. The aqueous dispersion of any one of Paragraphs A1 to A9, wherein the antiviral therapeutic agents and the one or more compatibilizers together comprise a repetitive multi-drug motif structure.
A11. The aqueous dispersion of any one of Paragraphs A1 to A10, wherein the aqueous dispersion does not comprise a lipid layer excipient, a lipid bilayer excipient, a liposome, or a micelle.
A12. The aqueous dispersion of any one of Paragraphs A1 to A11, wherein the aqueous solvent is selected from a buffered aqueous solvent, saline, and an aqueous solution of 20 mM sodium bicarbonate and 0.45 wt % to 0.9 wt % NaCl.
A13. The aqueous dispersion of any one of Paragraphs A1 to A12, wherein the aqueous dispersion comprises the antiviral therapeutic agent composition in an amount of 10 wt % or more and 25 wt % or less.
A14. The aqueous dispersion of any one of Paragraphs A1 to A13, in the form of a suspension.
A15. A method of treating diseases caused by retroviruses, comprising:
parenterally administering to a subject in need thereof, at a frequency of at most one dose every 2 weeks, an injectable aqueous dispersion of any one of Paragraphs A1 to A14.
A16. The method of Paragraph A15, wherein the treatment for diseases caused by retroviruses includes the treatment of acquired immune deficiency syndrome or an HIV infection.
A17. The method of Paragraph A15 or Paragraph A16, comprising parenterally administering the aqueous dispersion to the subject at a frequency of at most one dose per every 3 weeks.
A18. The method of any one of Paragraphs A15 to A17, comprising parenterally administering the aqueous dispersion to the subject at a frequency of at most one dose per every 4 weeks.
A19. The method of any one of Paragraphs A15 to A18, comprising intravenously administering the aqueous dispersion to the subject.
A20. The method of any one of Paragraphs A15 to A19, comprising subcutaneously administering the aqueous dispersion to the subject.
A21. The method of any one of Paragraphs A15 to A20, wherein the aqueous dispersion exhibits a 25- to 50-fold higher exposure of each antiviral therapeutic agent in non-human primates, when administered subcutaneously, compared to the exposure of each freely solubilized individual therapeutic agent.
A22. The method of any one of Paragraphs A15 to A21, wherein each therapeutic agent in the combination of therapeutic agents of the aqueous dispersion has a terminal half-life greater than the terminal half-life of each freely solubilized individual therapeutic agent.
A23. A powder composition comprising a combination of antiviral therapeutic agents selected from:
   dolutegravir, lamuvidine, and tenofovir and prodrugs thereof;
   efavirenz, lopinavir, and tenofovir and prodrugs thereof;
   lopinavir, ritonavir, lamuvidine, tenofovir and prodrugs thereof;
   efavirenz, tenofovir disoproxil fumarate, and emtricitabine (FTC);
   dolutegravir, tenofovir disoproxil fumarate, and emtricitabine;
   dolutegravir, lamuvidine, and tenofovir disoproxil fumarate;
   dolutegravir, lamuvidine, and abacavir;
   dolutegravir, lamuvidine, tenofovir and prodrugs thereof, and rilpivirine; and
the powder composition further comprising one or more compatibilizers comprising a lipid, a lipid conjugate, or a combination thereof;
wherein the powder composition exhibits a therapeutically effective plasma concentration of the combination of antiviral therapeutic agents for 2 or more weeks.
A24. The powder composition of Paragraph A23, wherein the one or more compatibilizers are selected from 1,2-distearoyl-sn-glycero-3-phosphocholine, 1,2-distearoyl-sn-glycero-3-phosphoethanolamine-N-[poly(ethylene glycol)2000], and a combination thereof.
A25. The powder composition of Paragraph A23 or Paragraph A24, wherein the combination of antiviral therapeutic agents is efavirenz, lopinavir, and tenofovir and prodrugs thereof at a molar ratio of about 0.8:1:15.
A26. The powder composition of Paragraph A23 or Paragraph A24, wherein the combination of antiviral therapeutic agents comprises tenofovir and prodrugs thereof: lamuvidine:dolutegravir at a molar ratio of from about 15:15:15.3 to about 21:26.2:14.4.
A27. The powder composition of Paragraph A23 or Paragraph A24, wherein the combination of antiviral therapeutic agents is selected from:
lopinavir, ritonavir, lamuvidine, and tenofovir and prodrugs thereof at a molar ratio of about 4:1:4:5; and
dolutegravir, lamuvidine, tenofovir and prodrugs thereof, and rilpivirine at a molar ratio of about 1:1:1:0.5.
A28. The powder composition of Paragraph A26 or Paragraph A27, wherein the composition exhibits a therapeutically effective plasma concentration of the combination of antiviral therapeutic agents for 3 or more weeks.
A29. The powder composition of Paragraph A26 or Paragraph A27, wherein the composition exhibits a therapeutically effective plasma concentration of the combination of antiviral therapeutic agents for 4 or more weeks.
A30. The powder composition of any one of Paragraphs A23 to A29, wherein the therapeutic agents and the one or more compatibilizers together form an organized composition.
A31. The powder composition of any one of Paragraphs A23 to A30, wherein the therapeutic agents and the one or more compatibilizers together comprise a long-range order in the form of a repeating pattern.
A32. The powder composition of any one of Paragraphs A23 to A31, wherein the therapeutic agents and the one or more compatibilizers together comprise a repetitive multi-drug motif structure.
A33. The powder composition of any one of Paragraphs A23 to A32, wherein the composition remains stable when stored at 25° C. for at least 2 weeks.
A34. The powder composition of any one of Paragraphs A23 to A33, wherein the composition does not comprise an amorphous solid dispersion.
A35. The powder composition of any one of Paragraphs A23 to A34, wherein the composition comprises a phase transition temperature different from the transition temperature of each individual antiviral therapeutic agent when assessed by differential scanning calorimetry.
A36. The powder composition of any one of Paragraphs A23 to A35, wherein the composition is in the form of homogeneous distribution of each individual antiviral therapeutic agent when viewed by scanning electron microscopy.
A37. The powder composition of any one of Paragraphs A23 to A36, wherein the composition comprises each antiviral therapeutic agent in an amount of 2 wt % or more and 20 wt % or less.
A38. The powder composition of any one of Paragraphs A23 to A37, wherein the composition comprises the one or more compatibilizers in an amount of 20 wt % or more and 95 wt % or less.
A39. The powder composition of any one of Paragraphs A23 to A38, comprising a molar ratio of therapeutic agents to the one or more compatibilizers of from 30:115 to 71:40.
A40. The powder composition of any one of Paragraphs A23 to A39, comprising particles having an average dimension of from 100 nm to 10 μm.

While illustrative embodiments have been illustrated and described, it will be appreciated that various changes can be made therein without departing from the spirit and scope of the invention.

1. An injectable aqueous dispersion, comprising:
(a) an aqueous solvent;
(b) an antiviral therapeutic agent composition dispersed in the aqueous solvent to provide the injectable aqueous dispersion, the antiviral therapeutic agent composition comprising a combination of antiviral therapeutic agents selected from:
(i) dolutegravir, lamivudine, and tenofovir and prodrugs thereof;
(ii) efavirenz, lopinavir, and tenofovir and prodrugs thereof;
(iii) lopinavir, ritonavir, lamivudine, tenofovir and prodrugs thereof;
(iv) efavirenz, tenofovir disoproxil fumarate, and emtricitabine;
(v) dolutegravir, tenofovir disoproxil fumarate, and emtricitabine;
(vi) dolutegravir, lamivudine, and tenofovir disoproxil fumarate;
(vii) dolutegravir, lamivudine, and abacavir; and
(viii) dolutegravir, lamivudine, tenofovir and prodrugs thereof, and rilpivirine; and
(c) one or more compatibilizers comprising a lipid, a lipid conjugate, or a combination thereof;
wherein the injectable aqueous dispersion exhibits a therapeutically effective plasma concentration of the combination of antiviral therapeutic agents for 2 or more weeks.

2. The aqueous dispersion of claim 1, wherein the one or more compatibilizers are selected from 1,2-distearoyl-sn-glycero-3-phosphocholine, 1,2-distearoyl-sn-glycero-3-phosphoethanolamine-N-[poly(ethylene glycol)2000], and a combination thereof.

3. The aqueous dispersion of claim 1, wherein the combination of antiviral therapeutic agents is efavirenz, lopinavir, and tenofovir and prodrugs thereof at a molar ratio of about 0.8:1:15.

4. The aqueous dispersion of claim 1, wherein the combination of antiviral therapeutic agents comprises tenofovir and prodrugs thereof: lamivudine:dolutegravir at a molar ratio of from about 15:15:15.3 to about 21:26.2:14.4.

5. The aqueous dispersion of claim 1, wherein the combination of antiviral therapeutic agents is selected from:
(i) lopinavir, ritonavir, lamivudine, and tenofovir and prodrugs thereof at a molar ratio of about 4:1:4:5; and
(ii) dolutegravir, lamivudine, tenofovir and prodrugs thereof, and rilpivirine at a molar ratio of about 1:1:1:0.5.

6-10. (canceled)

11. The aqueous dispersion of claim 1, wherein the aqueous dispersion does not comprise a lipid membrane, a bilayer, a liposome, or a micelle.

12. (canceled)

13. The aqueous dispersion of claim 1, wherein the aqueous dispersion comprises the antiviral therapeutic agent composition in an amount from 10 wt % to 25 wt %.

14. (canceled)

15. A method of treating a disease caused by a viral pathogen, comprising:
parenterally administering to a subject in need thereof, at a frequency of at most one dose every 2 weeks, an injectable aqueous dispersion of claim 1.

16. The method of claim 15, wherein the disease caused by the viral pathogen is acquired immune deficiency syndrome or an HIV infection.

17-18. (canceled)

19. The method of claim 15, comprising intravenously administering the aqueous dispersion to the subject.

20. The method of claim 15, comprising subcutaneously or intramuscularly administering the aqueous dispersion to the subject.

21-22. (canceled)

23. A powder composition comprising a combination of antiviral therapeutic agents selected from:
(i) dolutegravir, lamivudine, and tenofovir and prodrugs thereof;
(ii) efavirenz, lopinavir, and tenofovir and prodrugs thereof;
(iii) lopinavir, ritonavir, lamivudine, tenofovir and prodrugs thereof;
(iv) efavirenz, tenofovir disoproxil fumarate, and emtricitabine;
(v) dolutegravir, tenofovir disoproxil fumarate, and emtricitabine;
(vi) dolutegravir, lamivudine, and tenofovir disoproxil fumarate;
(vii) dolutegravir, lamivudine, and abacavir; and
(viii) dolutegravir, lamivudine, tenofovir and prodrugs thereof, and rilpivirine; and
one or more compatibilizers comprising a lipid, a lipid conjugate, or a combination thereof;
wherein the powder composition exhibits a therapeutically effective plasma concentration of the combination of antiviral therapeutic agents for 2 or more weeks.

24. The powder composition of claim 23, wherein the one or more compatibilizers are selected from 1,2-distearoyl-sn-glycero-3-phosphocholine, 1,2-distearoyl-sn-glycero-3-phosphoethanolamine-N-[poly(ethylene glycol)2000], and a combination thereof.

25. The powder composition of claim 23, wherein the combination of antiviral therapeutic agents is efavirenz, lopinavir, and tenofovir and prodrugs thereof at a molar ratio of about 0.8:1:15.

26. The powder composition of claim 23, wherein the combination of antiviral therapeutic agents comprises tenofovir and prodrugs thereof: lamivudine:dolutegravir at a molar ratio of from about 15:15:15.3 to about 21:26.2:14.4.

27. The powder composition of claim 23, wherein the combination of antiviral therapeutic agents is selected from:
lopinavir, ritonavir, lamivudine, and tenofovir and prodrugs thereof at a molar ratio of about 4:1:4:5; and
dolutegravir, lamivudine, tenofovir and prodrugs thereof, and rilpivirine at a molar ratio of about 1:1:1:0.5.

28-32. (canceled)

33. The powder composition of claim 23, wherein the composition remains stable when stored at 25° C. for at least 2 weeks.

34-36. (canceled)

37. The powder composition of claim 23, wherein the composition comprises each antiviral therapeutic agent in an amount from 2 wt % to 20 wt %.

38. The powder composition of claim 23, wherein the composition comprises the one or more compatibilizers in an amount from 20 wt % to 95 wt %.

39. The powder composition of claim 23, comprising a molar ratio of therapeutic agents to the one or more compatibilizers of from 30:115 to 71:40.

40. (canceled)
